(19) United States
(12) Patent Application Publication
(10) Pub. No.: US 2022/0002792 A1
(43) Pub. Date: Jan. 6, 2022

(54) ACCELERATED POLYMERASE CHAIN REACTION

(71) Applicant: Igor V. Kutyavin, Woodinville, WA (US)

(72) Inventor: Igor V. Kutyavin, Woodinville, WA (US)

(21) Appl. No.: 17/290,166

(22) PCT Filed: Oct. 28, 2019
(86) PCT No.: PCT/US2019/058373

§ 371 Date: Apr. 29, 2021

Related U.S. Application Data
(60) Provisional application No. 62/752,283, filed on Oct. 29, 2018.
(60) Provisional application No. 62/843,193, filed on May 3, 2019.

(51) Int. Cl. C12Q 1/6848; C12P 19/34; C12Q 1/686

(52) U.S. Cl. CPC C12Q 1/6848; C12Q 1/686; C12P 19/34

(57) ABSTRACT

Provided are methods for accelerated PCR wherein the amount of the amplified material is more than doubled within each of a plurality of successive cycles. The methods comprise the use of at least three primers and an incubation step at a sufficient temperature (acceleration temperature) that is less than an inter-cycle PCR denaturation temperature. In the invention embodiment, some target-specific primer extension products produced in a particular PCR cycle are amplified twice in each successive PCR cycle, once prior to incubating at the sufficient temperature, and once thereafter. Also provided are kits comprising at least three target-specific oligonucleotides configured to provide for accelerated PCR.

### INCORPORATION OF SEQUENCE LISTING

The content of the text file named “0067898_011 WO0_ST25.txt,” which was created on Oct. 25, 2019, and is 3.68 KB in size, is hereby incorporated by reference in its entirety.

### FIELD OF THE INVENTION

Aspects of the invention relate generally to polymerase chain reaction (PCR) methods, and more particularly to highly productive, accelerated PCR methods wherein the amount of the amplified nucleic acid sequences is more than doubled during each of a plurality of cycles of the PCR. Additional aspects relate to PCR kits configured to provide for accelerated PCR.

### BACKGROUND

Polymerase chain reaction (PCR) (see, e.g., U.S. Pat. Nos. 4,683,195 and 4,683,202 to Mullis, K. B., 1987) continues to be the most commonly used technology for amplification of nucleic acids in research laboratories as well as in commercial applications. As practiced, the amount of amplified DNA material can be doubled in each PCR cycle. The specificity, sensitivity and reaction speed for nucleic acid detection could be improved, however, by increasing the PCR amplification power so that the amount of amplified material more than doubles (e.g., triples, quadruples or greater) during average PCR cycles.

### BRIEF DESCRIPTION OF THE DRAWINGS

FIG. 1A illustrates, according to particular exemplary aspects of the present invention, amplifying a target nucleic acid sequence (e.g., DNA) in a sample using three oligonucleotide primers P1, P2, and P3 during a first PCR cycle. Primer P3 hybridizes to a complementary primer binding site on the second strand of the target sequence at a position 5′ upstream from the hybridized primer P2.

FIG. 1B illustrates, according to particular exemplary aspects of the present invention, amplifying the target nucleic acid sequence using the three oligonucleotide primers P1, P2 and P3 during a next, successive cycle of the PCR (second PCR cycle).

FIG. 1C shows the amplification products present in reaction mixture at the beginning (left) and at the end (right) of further successive cycle of the PCR (third cycle) incorporating all four steps (step 1 through step 4).

FIG. 2A illustrates, according to particular exemplary aspects of the present invention, amplifying, in a first PCR cycle, the target nucleic acid sequence of FIG. 1 using four oligonucleotide primers P1, P2, P3 and P4. Similar to FIG. 1A, FIG. 2A shows the amplification scheme for two complementary strands of the target nucleic acid sequence (solid lines) with adjacent contiguous (indefinite ends) (dashed lines).

FIG. 2B shows a next, successive cycle of the PCR. The P2 primer extension product with the indefinite 3′-sequence incorporates P1 and P4 primer binding sites (reaction scheme shown on the left of FIG. 2B), whereas the P1 primer extension product with its indefinite 3′-sequence incorporates P2 and P3 binding sites (reaction scheme shown on the right of FIG. 2B). As illustrated in FIG. 2B for convenience, the PCR cycle is presented as being divided into two paralleled processes, which in analogy to the PCR cycle of FIG. 1B, proceed through steps 1, 2, 3A, 3B and 4.

FIG. 3 shows, according to particular exemplary aspects of the present invention, a fragment of M13mp18 vector sequence (target DNA sequence, SEQ ID NO:6) and eight 2′-deoxyribo oligonucleotide primers (SEQ ID NOS:1-5, 7-9) used in the working Examples provided herein.

FIG. 4 shows, according to particular exemplary aspects of the present invention, sequences of oligonucleotides (SEQ ID NOS:10-12) used in melting experiments described in detail herein in Example 2. The graph in FIG. 4 shows the results of these melting experiments provided in the form of a first derivative (“dF/dT”) of the corresponding fluorescence melting curves (Y-axis) plotted against the reaction temperature on the X-axis.

FIGS. 5A and 5B show, according to particular exemplary aspects of the present invention, results of EvaGreen™ fluorescence monitoring during conventional PCR in comparison with accelerated PCR using a three-primer embodiment of the invention according to the reaction scheme of FIGS. 1A-1C (SEQ ID NO:1 as P1 primer, SEQ ID NO:2 as P2 primer and SEQ ID NO:3 as P3 primer, FIG. 3).

FIGS. 6A and 6B show, according to particular exemplary aspects of the present invention, results of EvaGreen™ fluorescence monitoring during conventional PCR, in comparison with accelerated PCR using a three-primer method embodiment of the invention according to the reaction scheme of FIGS. 1A-1C. The experiments of FIGS. 6A and 6B are identical in all aspects, including the reaction composition, PCR setup and data presentation to those shown in FIGS. 5A and 5B, but primer SEQ ID NO:3 used in FIGS. 5A and 5B as the third P3 primer was replaced by primer SEQ ID NO:4 (see FIG. 3) as indicated.

FIGS. 7A and 7B show, according to particular exemplary aspects of the present invention, results of EvaGreen™ fluorescence monitoring during accelerated PCR using a four-primers' method embodiment of the invention according to the reaction scheme of FIGS. 2A and 2B (SEQ ID NOS:1-4, see FIG. 3) and a PCR time/temperature profile incorporating step 4 as indicated. The reaction composition and PCR setup as well as the data presentation are otherwise identical to those shown in FIGS. 5 and 6. Fluorescence threshold for each curve of FIG. 7A was determined and plotted versus logarithm of the target loads in FIG. 7B. The slope coefficients of the linear equations were used to calculate the PCR amplification power in this particular case.

FIGS. 8A and 8B show, according to particular exemplary aspects of the present invention, detection of accelerated PCR amplified material in real time using a FRET-labelled P2 primer. The reaction mixtures in FIG. 8A had the same composition and time/temperature profile as those marked by filled circles (•) in FIG. 5A, but P2 primer SEQ ID NO:2 was replaced by its FRET-labelled analog SEQ ID NO:5 (see structures in FIG. 3) and EvaGreen™ fluorescent dye was omitted. Fluorescence threshold for each curve of FIG. 8A was determined and plotted versus logarithm of the target loads in FIG. 8B. The slope coefficients of the linear equations were used to calculate the PCR amplification power in this particular case.

FIG. 9 illustrates, according to particular exemplary aspects of the present invention, amplifying a target nucleic acid sequence (e.g., DNA) in a sample using three oligonucleotide primers P1, P2, and P3 during a second PCR cycle, wherein the primer P3 binding site overlaps the binding site of P2 primer. Amplification components produced during the first cycle of this method embodiment along with the original first and second target strands are shown in the dashed box at the upper left corner.

FIGS. 10A and 10B show, according to particular exemplary aspects of the present invention, results of EvaGreen™ fluorescence monitoring during conventional PCR in comparison with accelerated PCR using a three-primer embodiment of the invention (SEQ ID NO:1 as P1 primer, SEQ ID NO:2 as P2 primer and SEQ ID NO:12 as P3 primer, FIGS. 3 and 4). In this embodiment, the 5′-sequence of the P3 primer binding site is located immediately adjacent to the 3′-sequence of P2 primer binding site.

FIGS. 11A and 11B show, according to particular exemplary aspects of the present invention, results of EvaGreen™ fluorescence monitoring during conventional PCR in comparison with accelerated PCR using a three-primer embodiment of the invention according to the reaction scheme of FIG. 9 (SEQ ID NO:7 as P1 primer, SEQ ID NO:8 as P2 primer and SEQ ID NO:9 as P3 primer, FIG. 3), wherein the P3 primer binding site overlaps the binding site of P2 primer.

FIG. 12 illustrates, according to particular exemplary aspects of the present invention, amplifying a target nucleic acid sequence (e.g., DNA) in a sample using three oligonucleotide primers P1, P2, and P3 during a second PCR cycle wherein P2 and P3 primers are covalently coupled to each other at their 5′-ends through a linker. Amplification components produced during the first cycle of this embodiment along with the original first and second target strands are shown in the dashed box at the upper left corner.

FIG. 13A shows, according to particular exemplary aspects of the present invention, a fragment of M13mp18 vector sequence (target DNA sequence, SEQ ID NO:15) and two 2′-deoxyribo oligonucleotide primers (SEQ ID NO:1) and a composite oligonucleotide comprising two primers (SEQ ID NOS:13 and 14) coupled through their 5′-ends used in the PCR experiments of FIGS. 13B and 13C, wherein the reverse primer SEQ ID NO:13 corresponds to an oligonucleotide primer wherein primers P2 and P3 are coupled at their 5′-ends through a polymer linker.

FIGS. 13B and 13C show, according to particular exemplary aspects of the present invention, results of EvaGreen™ fluorescence monitoring during conventional PCR in comparison with accelerated PCR protocol according to the reaction scheme of FIG. 12 (SEQ ID NO:1 as P1 primer, SEQ ID NOS:13 and 14 as P2 and P3 primers coupled at their 5′-ends, FIG. 13A), wherein the P3 primer binding site is located at a position 5′ upstream from the hybridized P2 primer. The fluorescence threshold for each curve of FIG. 13B was determined and plotted versus the logarithm of the target loads in FIG. 13C. The slope coefficients of the linear equations were used to calculate the PCR amplification power in this embodiment.

### SUMMARY OF EXEMPLARY EMBODIMENTS OF THE INVENTION

Embodiments of the disclosure can be described in view of the following clauses:

1. A method for accelerated polymerase chain reaction (PCR) amplification, comprising performing PCR in a suitable reaction mixture containing DNA polymerase, a nucleic acid target sequence and at least three oligonucleotide primers each complementary to a respective primer binding site of the target sequence and each present in excess relative to the target sequence, wherein at least one cycle of the PCR includes:
producing a first primer (P1) extension product hybridized to a first strand of the target sequence and having a binding site for a second oligonucleotide primer (P2) and for a third oligonucleotide primer (P3);
producing a P2 extension product hybridized to a second strand of the target sequence and having a P1 primer binding site at its 3′ end; and
incubating the reaction mixture at a temperature sufficient to initiate thermal melting of the hybridized P2 extension product, and producing, at the sufficient temperature, a full-length P3 primer extension product hybridized to the second strand of the target sequence and having a sequence complementary to the P2 binding site, and having a P1 binding site at its 3′ end, wherein the P2 primer extension product is not hybridized to the second strand of the target sequence and is accessible to be primed by another P1 primer, and wherein the sufficient temperature is less than a denaturation temperature used to initiate a next, successive cycle of the PCR.
2. The method of clause 1, wherein the at least one cycle of the PCR comprises: hybridizing the P1 primer to the first strand of the target sequence and extending the hybridized P1 primer to provide the P1 primer extension product hybridized to the first strand of the target sequence and having the binding site for the P2 primer and for the P3 primer;
hybridizing the P2 primer to the second, complementary strand of the target sequence and extending the hybridized P2 primer to provide the P2 primer extension product hybridized to the second strand of the target sequence and having the P1 primer binding site at its 3′ end;
hybridizing the P3 primer to the second strand of the target sequence at a position 5′ upstream from the hybridized P2 primer extension product, and extending the hybridized P3 primer toward the 5′-end of the hybridized P2 primer extension product to provide a partial P3 primer extension product hybridized to the second strand of the target sequence and lacking the P1 primer binding site at its 3′ end; and
incubating the reaction mixture at the temperature sufficient to initiate thermal melting of the hybridized P2 primer extension product, wherein the hybridized partial P3 primer extension product has a thermal stability sufficient to provide for its further extension at the sufficient temperature, and further extending the hybridized partial P3 primer extension product to provide the full-length P3 primer extension product hybridized to the second strand of the target sequence.
3. The method of clause 1, wherein the at least one cycle of the PCR comprises: hybridizing the P1 primer to the first strand of the target sequence and extending the hybridized P1 primer to provide the P1 primer extension product hybridized to the first strand of the target sequence and having the binding site for the P2 primer and for the P3 primer;
hybridizing the P2 primer to the second, complementary strand of the target sequence and extending the hybridized P2 primer to provide the P2 primer extension product hybridized to the second strand of the target sequence and having the P1 primer binding site at its 3′ end;
incubating the reaction mixture at the temperature sufficient to initiate thermal melting of the hybridized P2 primer extension product; and
hybridizing the P3 oligonucleotide primer to the second strand of the target sequence at a position immediately adjacent the 3′-end of the P2 primer binding site, or at a position overlapping the P2 primer binding site, and extending, at the sufficient temperature, the hybridized P3 primer to provide the full-length P3 primer extension product hybridized to the second strand of the target sequence.
4. The method of any of clauses 1-3, further comprising, in the at least one cycle of the PCR, hybridizing another P1 primer to the P2 primer extension product not hybridized to the second strand of the target sequence, and extending the hybridized other P1 primer to provide a P1/P2 double-stranded extension product having P1 and P2 primer binding sites at its 3′ ends.
5. The method of clause 4, further comprising, after forming the P1/P2 double-stranded extension product, incubating the reaction mixture at a denaturation temperature greater than the sufficient temperature to denature all hybridized primer extension products including the P1 primer extension product having the P2 and the P3 primer binding sites, the full-length P3 primer extension product, and the P1/P2 double-stranded extension product.
6. The method of clause 5, further comprising in a next, successive cycle of the PCR:
hybridizing additional P1 and P2 primers to respective primer binding sites of the denatured primer extension products from the preceding cycle, including to the respective primer binding sites of the denatured strands of the P1 primer extension product, of the full-length P3 primer extension product, and of the P1/P2 double-stranded extension product;
extending the hybridized additional P1 and P2 primers to provide additional hybridized P1 and P2 primer extension products, including an additional P2 primer extension product hybridized to the P1 primer extension product, an additional P1 primer extension product hybridized to the P3 primer extension product, and additional P1/P2 double-stranded extension products having additional P1 and P2 primer binding sites at their 3′ ends;
incubating the reaction mixture at the sufficient temperature to initiate thermal melting of the additional hybridized P2 primer extension products, including of the additional P2 primer extension product hybridized to the P1 primer extension product, and of the additional P1/P2 double-stranded extension products;
hybridizing yet additional P1 and P2 primers to respective primer binding sites of the thermally-melted additional P2 primer extension products, including to respective primer binding sites of the thermally melted strands of the additional P2 primer extension product hybridized to the P1 primer extension product, and of the additional P1/P2 double-stranded extension products; and
extending the hybridized yet additional P1 and P2 primers to provide yet additional P1/P2 double-stranded extension products having yet additional P1 and P2 primer binding sites at their 3′ ends, wherein the P1/P2 double-stranded extension product produced in the preceding at least one cycle of the PCR is amplified twice in this successive cycle of the PCR, once prior to incubating the reaction mixture at the sufficient temperature, and once thereafter.
7. The method of clause 6, wherein at least one of the yet additional P1/P2 double-stranded extension products is derived from the P1 primer extension product of the preceding at least one cycle of the PCR.
8. The method of clauses 6 or 7, comprising hybridizing additional P3 primers to respective primer binding sites of the denatured primer extension products from the preceding at least one PCR cycle that have P3 primer binding sites, and extending, at the sufficient temperature, the hybridized additional P3 primer extension products to produce additional full-length P3 primer extension products.
9. The method of clause 8, further comprising, incubating the reaction mixture at the denaturation temperature to denature all hybridized primer extension products.
10. The method of clause 9, further comprising, in a further successive cycle of the PCR, twice amplifying at least one, more than one, or substantially all of the yet additional P1/P2 double-stranded extension products.
11. The method of any of clauses 4-10, wherein, in the at least one cycle of the PCR, the hybridizing another P1 primer to the P2 primer extension product not hybridized to the second strand of the target sequence is performed at a lower reaction temperature than the sufficient temperature.
12. The method of any of clauses 2, 4-11, wherein in the at least one cycle of the PCR, hybridizing the P3 primer to the second strand of the target sequence is performed at an identical, different, or lower reaction temperature than a temperature used for hybridizing the P2 primer to the second strand of the target sequence.
13. The method of any of clauses 1-12, wherein, in the at least one cycle of the PCR, the hybridized P3 primer, or the hybridized partial P3 primer extension product, has a greater thermal stability than that of the hybridized P2 primer extension product having a P1 primer binding site at its 3′ end.
14. The method of any of clauses 1-13, wherein upon completion of the PCR, the number of P2 primer extension products is greater than that of the P3 primer extension products, at least in part because the P2 primer extension products are amplified twice in one or in each of a plurality of cycles of the PCR.
15. The method of any of clauses 1-14, wherein upon completion of the PCR, the ratio of the number of P2 primer extension products to that of the full-length P3 primer extension products is determined, at least in part, by at least one of: the distance between the second and third primer binding sites on the second strand of the target sequence; the relative concentrations of the second and third primers; or by the relative thermal stability of the complementary duplexes of the second and the third primers with their respective binding sites.
16. The method of any of clauses 1-15, wherein the concentration of the P2 primer is greater than that of the P3 primer.
17. The method of any of clauses 2, 4-16, wherein the thermal stability of the complementary duplex of the P2 primer with its binding site is greater than that of the complementary duplex of the P3 primer with its binding site.
18. The method of any of clauses 2, 4-17, further comprising a fourth oligonucleotide primer (P4) complementary to a respective primer binding site of the target sequence and present in excess relative to the target sequence, wherein the at least one cycle of the PCR includes hybridizing the P4 primer to the first strand of the target sequence at a position 5′ upstream from the hybridized P1 primer extension product, and extending the hybridized P4 primer toward the 5′-end of the hybridized P1 primer extension product to provide a partial P4 primer extension product hybridized to the first strand of the target sequence and lacking a P2 primer binding site at its 3′ end, wherein the sufficient temperature is sufficient to initiate thermal melting of the hybridized P1 primer extension product, and wherein the hybridized P4 primer extension product has a thermal stability sufficient to provide for its further extension at the sufficient temperature; and further extending the hybridized P4 primer extension product to produce a full-length P4 primer extension product hybridized to the first strand of the target sequence and having a P2 primer binding site, and wherein the P1 primer extension product is not hybridized to the first strand of the target sequence, and is accessible to priming by another P2 oligonucleotide primer.
19. The method of any of clauses 1-18, wherein the distance, in nucleotides, between the 5′ end of P1 primer binding site on the first strand and the 5′ end of the P2 primer binding site on the second strand is less than 20, less than 15, less than 10, less than 5, less than 4, less than 3, less than 2, 1, or 0, or is a value in the range of 0 to 20, or in any subrange thereof.
20. The method of clause 19, wherein the distance is 0 to 3 nucleotides.
21. The method of any of clauses 1-20, wherein an amplification power of at least 2.2 is provided.
22. The method of clause 21 wherein an amplification power of at least 2.5 is provided.
23. The method of any of clauses 1-22, wherein the P1 primer, the P2 primer, or both incorporate at least one polymerase-compatible duplex-destabilizing modification.
24. The method of any of claims 1-23, wherein the P3 primer incorporates at least one polymerase-compatible duplex-stabilizing modification.
25. The method of any of clauses 18-24, wherein the P1 primer, the P2 primer, or both incorporate at least one polymerase-compatible duplex-destabilizing modification, and wherein the P3 primer, the P4 primer, or both incorporate at least one polymerase-compatible duplex-stabilizing modification.
26. The method of any of clauses 1-25, wherein the amplification products are detected.
27. The method of clause 26, wherein the amplification and detection reactions are performed simultaneously, in real time.
28. The method of any of clauses 1-27, further comprising determining the amount of the target nucleic acid in or from a sample.
29. The method of any of clauses 1-28, wherein the reaction mixture further comprises a detectable label.
30. The method of clause 29, wherein the detectable label comprises a fluorescent label.
31. The method of clause 30, wherein the reaction mixture comprises an oligonucleotide probe labeled with two dyes that are in FRET interaction, and wherein duplex formation of the probe with products of extension of the P1 or the P2 primers disrupts FRET resulting in a detectable signal.
32. The method of clause 30, wherein at least one of the P1 and the P2 primers is labeled with two dyes that are in a FRET interaction, and wherein hybridization and extension of the at least one labeled primer during PCR disrupts the FRET interaction resulting in a detectable signal.
33. The method of any of clauses 1, 2, 4-32, wherein the P2 and the P3 primers are covalently coupled to each other.
34. The method of clause 33, wherein the P2 and the P3 primers are covalently coupled at their 5′-ends.
35. The method of clause 34, wherein the P2 and the P3 primers are coupled through a linker.
36. The method of clause 35, wherein the linker comprises a oligoethylene glycol moiety.
37. A PCR kit, comprising at least three oligonucleotide primers each complementary to a respective primer binding site of a target sequence, wherein a first oligonucleotide primer (P1) is complementary to a P1 primer binding site on a first strand of the target sequence, wherein the second oligonucleotide primer (P2) is complementary to a P2 primer binding site on a second, complementary strand of the target sequence to define a P1/P2 amplicon sequence of the target sequence, wherein the third oligonucleotide primer (P3) is complementary to a P3 primer binding site on the second strand of the target sequence, and wherein, relative to the target sequence, the sequences and relative positions of the P2 and third P3 binding sites on the second strand of the target sequence are configured such that thermal stability of a P3 primer, or of a P3 primer extension product extending to the 3′-end of the second primer binding site is greater than that of a P2 primer extension product having a P1 primer binding site at its 3′-end.
38. The PCR kit of clause 37, wherein the P3 primer binding site on the second strand of the target sequence is at a position 3′ downstream from the P2 primer binding site.
39. The PCR kit of clause 38, wherein the P2 and the P3 primers are covalently coupled to each other.
40. The PCR kit of clause 39, wherein the P2 and the P3 primers are covalently coupled at their 5′-ends.
41. The PCR kit of clause 40, wherein the P2 and the P3 primers are coupled through a linker.
42. The PCR kit of clause 41, wherein the linker comprises a oligoethylene glycol moiety.
43. The PCR kit of any of clauses 37-42, wherein the distance, in nucleotides, between the 5′ end of P1 primer binding site on the first strand and the 5′ end of the P2 primer binding site on the second strand is less than 20, less than 15, less than 10, less than 5, less than 4, less than 3, less than 2, 1, or 0, or is a value in the range of 0 to 20, or is a value in the range of 0 to 20, or in any subrange thereof.
44. The PCR kit of clause 43, wherein the distance is 0 to 3 nucleotides.
45. A PCR kit, comprising at least three oligonucleotide primers each complementary to a respective primer binding site of a target sequence, wherein a first oligonucleotide primer (P1) is complementary to a P1 primer binding site on a first strand of the target sequence, wherein a second oligonucleotide primer (P2) is complementary to a P2 primer binding site on a second, complementary strand of the target sequence to define an P1/P2 amplicon sequence of the target sequence, wherein a third oligonucleotide primer (P3) is complementary to a P3 primer binding site on the second strand of the target sequence, and wherein, relative to the target sequence, the distance, in nucleotides, between the 5′ end of P1 primer binding site on the first strand and the 5′ end of the P2 primer binding site on the second strand is less than 20, less than 15, less than 10, less than 5, less than 4, less than 3, less than 2, 1, or 0, or is a value in the range of 0 to 20, or in any subrange thereof.
46. The PCR kit of clause 45, wherein the distance is 0 to 3 nucleotides.
47. The PCR kit of any of clauses 45-46, wherein the P3 primer binding site on the second strand of the target sequence is at a position 3′ downstream from the P2 primer binding site.
48. The PCR kit of clause 47, wherein the P2 and the P3 primers are covalently coupled to each other.
49. The PCR kit of clause 48, wherein the P2 and the P3 primers are covalently coupled at their 5′-ends.
50. The PCR kit of clause 49, wherein the P2 and the P3 primers are coupled through a linker.
51. The PCR kit of clause 50, wherein the linker comprises a oligoethylene glycol moiety.
52. The PCR kit of any of clauses 45-51, wherein, relative to the target sequence, the sequences and relative positions of the P2 and the P3 primer binding sites on the second strand of the target sequence are such that thermal stability of a P3 primer, or of a P3 primer extension product extending to the 3′-end of the P2 primer binding site is greater than that of a P2 primer extension product having a P1 primer binding site at its 3′-end.

### DETAILED DESCRIPTION OF EXEMPLARY EMBODIMENTS

Terms and symbols of biochemistry, nucleic acid chemistry, molecular biology and molecular genetics used herein follow those of standard treaties and texts in the field (e.g., Sambrook, J., et al, 1989; Kornberg, A. and Baker, T., 1992; Gait, M. J., ed., 1984; Lehninger, A. L., 1975; Eckstein, F., ed., 1991, and the like). To facilitate understanding of particular exemplary aspects of the invention, a number of terms are discussed below.

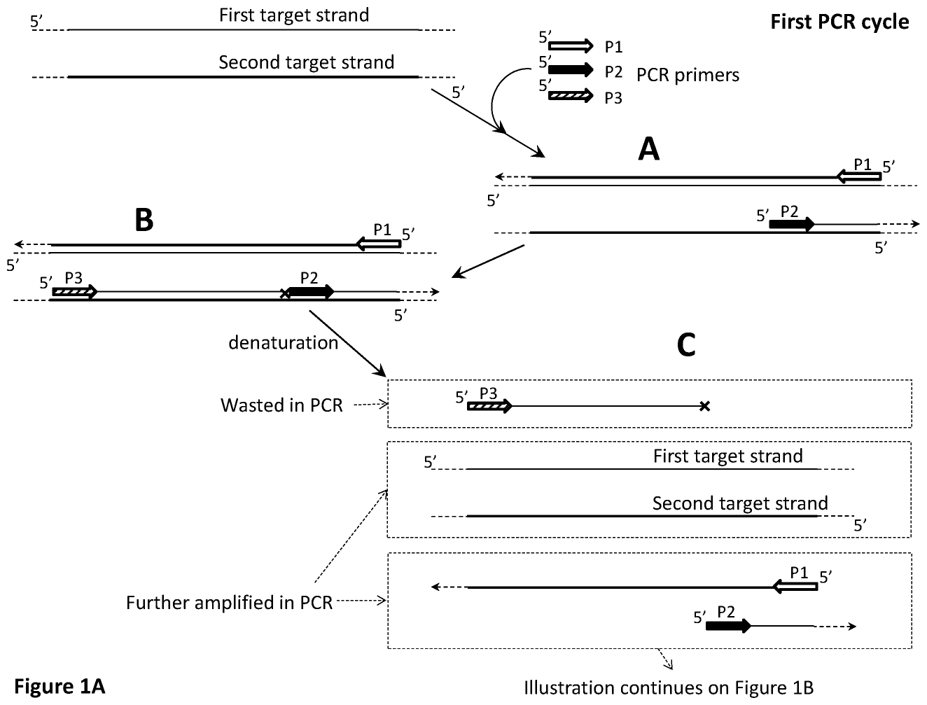

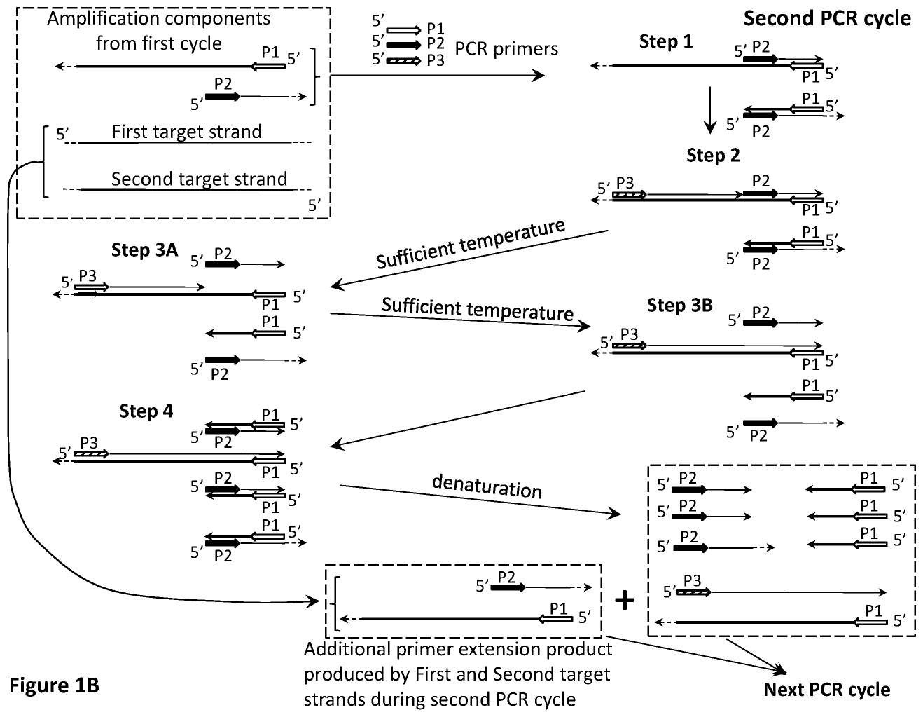

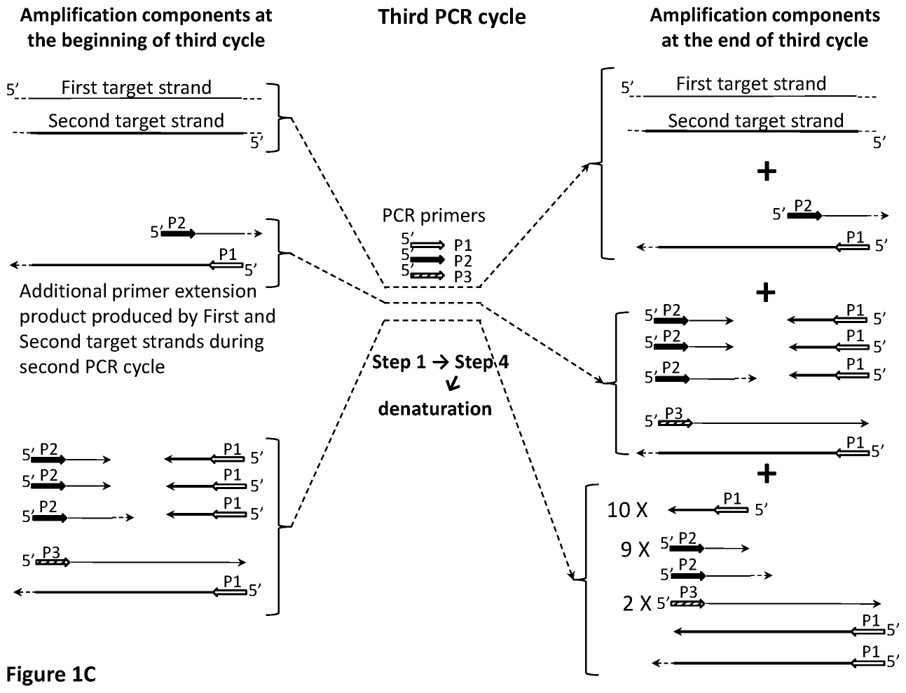

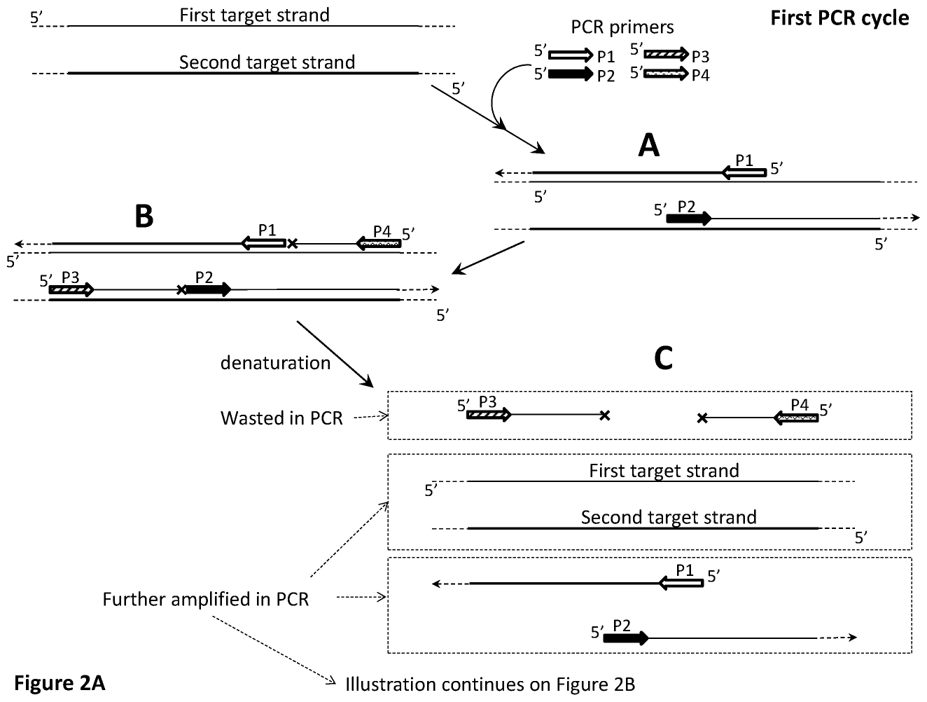

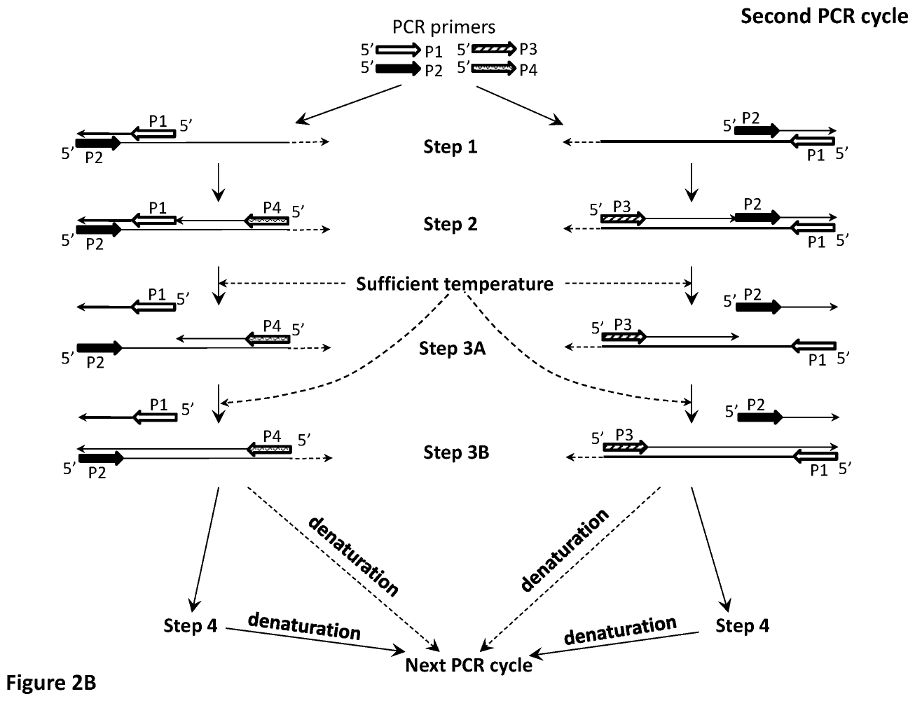

In methods of the invention, a target nucleic acid is amplified by PCR. “PCR” is an abbreviation of term “polymerase chain reaction,” the art-recognized nucleic acid amplification technology (e.g., U.S. Pat. Nos. 4,683,195 and 4,683,202, issued to Mullis, K. B.). The commonly used conventional PCR protocol employs two oligonucleotide primers, one for each strand, designed such that extension of one primer provides a template for the other primer in the next PCR cycle. Generally, a PCR reaction consists of repetitions (or cycles) of (i) a denaturation step which separates the strands of a double-stranded nucleic acid, followed by (ii) an annealing step, which allows primers to hybridize to positions flanking a sequence of interest, and then (iii) an extension step which extends the primers in a 5′ to 3′ direction, thereby forming a nucleic acid fragment complementary to the target sequence. Each of the above steps may be conducted at a different temperature using an automated thermocycler. The PCR cycles can be repeated as often (as many times) as desired resulting in an exponential accumulation of a target DNA amplicon fragment whose termini are usually defined by the 5′-ends of the primers used. Particular temperatures, incubation times at each step and rates of change between steps (temperature ramping rates) depend on many factors and examples can be found in numerous published protocols (e.g., McPherson, M. J., et al., 1991 and 1995). Although conditions of PCR can vary in a broad range, a double-stranded target nucleic acid is usually denatured at a temperature of >90.degree. C., primers are annealed at a temperature in the range of about 50-70.degree. C., and the extension is preferably performed in the 70.degree. C.-74.degree. C. range. The term “PCR” encompasses derivative forms of the reaction, including but not limited to, “RT-PCR,” “real-time PCR,” “asymmetric PCR,” “nested PCR,” “quantitative PCR,” “multiplexed PCR,” and the like. Cycles in PCR are separated from each other by a denaturation temperature or denaturation step at which usually all double-stranded products of the primers' extensions are melted. DNA amplification in PCR takes place at lower temperatures than denaturation, and it does not matter whether denaturation step is programed to start or end a PCR cycle. Target nucleic acid can be a fragment or contiguous portion of a very long double-stranded molecule, and therefore, prior to PCR cycling, the reaction protocols commonly incorporate an incubation at a denaturation temperature or greater for a sufficient time to render the polymer single stranded. The denaturation temperature does not need to be kept constant through all cycles of PCR. For example, after few initial cycles of PCR with accumulation of amplification products defined by the sequences of primers used, the denaturation temperature can be lowered such as only these products denature while the primer extension products with indefinite 3′-ends remain double-stranded. However, this is not recommended because this excludes the primer extension products with indefinite 3′-ends from the amplification process and can reduce the overall PCR amplification power including in the accelerated PCR methods of the invention described herein. In the methods, “target nucleic acid” or “nucleic acid of interest” refers to a nucleic acid or a fragment or contiguous portion of nucleic that is to be amplified and/or detected using methods of the present invention. For example, the target nucleic acid sequence is framed by sequences and/or binding sites of P1 and P3 primers in methods of FIG. 1 whereas in methods of FIG. 2 these “framing” primers are P3 and P4. Nucleic acids of interest (e.g., target sequences) can be of any size and sequence. Preferably, the nucleic acid is of a size that provides for amplification and/or detection thereof. Two or more target nucleic acids can be fragments or portions (e.g., separated or contiguous portions) of the same nucleic acid molecule. As used herein, target nucleic acids are different if they differ in nucleotide sequence by at least one nucleotide. Target nucleic acids can be single-stranded or double-stranded. When a nucleic acid of interest is double-stranded or presumed to be double-stranded, the term “target nucleic acid” refers to a specific sequence in either strand of double-stranded nucleic acid. Therefore, the full complement to any single stranded nucleic acid of interest is treated herein as the same (or complementary) target nucleic acid. When a target nucleic acid comprises only one strand, the primers are preferably selected such as P1 primer hybridized to that single-stranded target sequence. When target nucleic acids are double-stranded, they are rendered single stranded by any physical, chemical or biological approach before applying the methods of the invention. For example, double-stranded nucleic acid can be denatured at elevated temperature, e.g. 90-95° C. as was used in the examples provided herein. Nucleic acids incorporating the target nucleic acids' sequences may be derived from any organism or other source, including but not limited to prokaryotes, eukaryotes, plants, animals, and viruses, as well as synthetic nucleic acids. The target nucleic acids may be DNA, RNA, and/or variants thereof. Nucleic acids of interest can be isolated and purified from the sample sources before applying methods of the present invention. Preferably, the target nucleic acids are sufficiently free of proteins and any other substances interfering with primer-extension and/or detection reactions. Many methods, for example, as described in Ausubel, F. M., et al., eds., 1993; Walsh, P. S., et al., 1991; Boom, W. R., et al., 1993; Miller, S. A., et al., 1988, are available for the isolation and purification of nucleic acids of interest including commercial kits and specialty instruments. In a preferred embodiment, the target nucleic acid is DNA. In another embodiment, the target nucleic is RNA. Prior to applying the methods of the invention, a DNA copy (cDNA) of target RNA can be obtained using an oligonucleotide primer that hybridize to the target RNA, and extending of this primer in the presence of a reverse transcriptase and nucleoside 5′-triphosphates (dNTPs). The resulting DNA/RNA heteroduplex can then be rendered single-stranded using techniques known in the art, for example, denaturation at elevated temperatures. Alternatively, the RNA strand may be degraded in presence of RNase H nuclease. When the target nucleic acid is RNA, P1 primer of the invention (e.g. methods of FIG. 1) can be used as an RT-primer for a reverse transcriptase to initiate synthesis of a cDNA copy of the target nucleic acid. In the methods, “amplification” and “amplifying” target nucleic acids, in general, refers to a procedure wherein multiple copies of the nucleic acid of interest are generated in the form of DNA copies. The terms “amplicon” or “amplification product” refer to a primer-extension product or products of amplification that may be a population of polynucleotides, single- or double-stranded, that are replicated from either strand or both, or from one or more nucleic acids of interest. Regardless of the originating target nucleic acid strand and the amplicons state, e.g. double- or single-stranded, all amplicons which are usually homologous are treated herein as amplification products of the same target nucleic acid including the products of incomplete extension. For example, as illustrated in FIGS. 1A-C and 2A-B, the methods of the invention produce amplicons of different length. All these amplification products that are homologous in sequence to the original nucleic acid of interest are treated herein as target amplification products regardless of their length and amplified sequence. For example, as illustrated in FIGS. 1 and 2, certain primer extension products can incorporate only a fragment or portion of the target sequence wherein some of these products yet comprise sequences other than the target nucleic acid, due to their indefinite ends. All these products of primer extension are treated herein as target amplification products or target amplicons. In particular aspects, the term “homology” and “homologous” refers to a degree of identity between nucleic acids. There may be partial homology or complete homology.

In conventional PCR using two primers, the number of amplification products comprising target nucleic acid sequence can double in each consecutive cycle, if quantitative yield is achieved in primer annealing and extension reactions. Then the number or concentration (C) of target nucleic acid sequence in each PCR cycle can be calculated using a simple equation C=2ⁿ×C₀ wherein ‘n’ is the cycle number and ‘C₀’ is the initial target load in a sample or reaction. The term “target load” means initial concentration or number of molecules or “copies” of target nucleic acid sequences in a sample or PCR reaction.

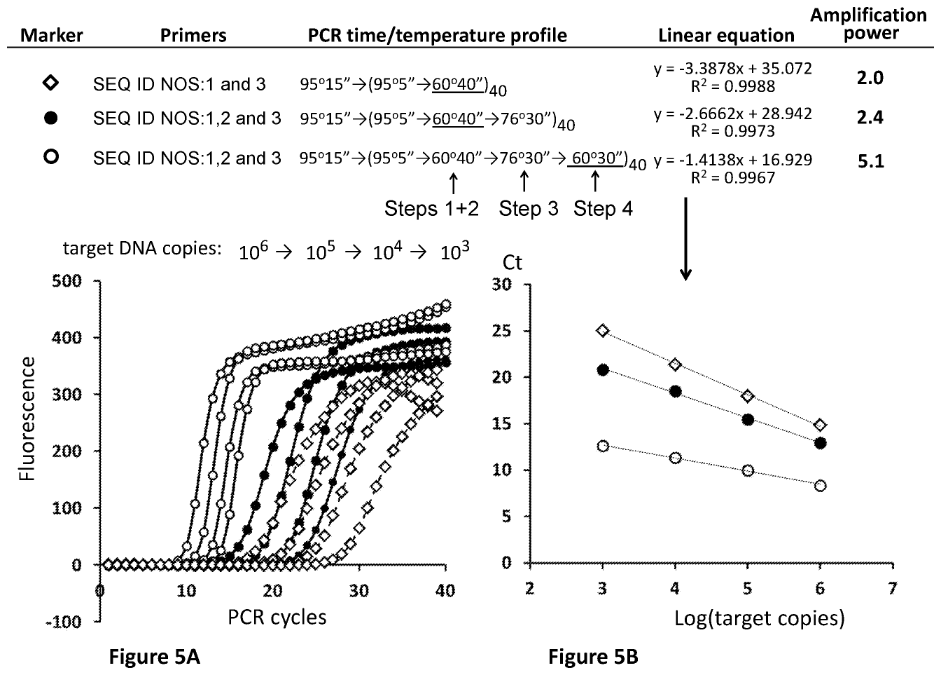

As used herein in the methods, the term “accelerated PCR” means a PCR method wherein the number of amplification products or molecules comprising target nucleic acid sequence can more than double in one or more, a plurality of, many, most, a majority of consecutive cycles. Similar to conventional PCR, in methods of the invention the number or concentration (C) of target nucleic acid sequences and target amplification products in each PCR cycle can be calculated using an exponential equation C=bⁿ×C₀ wherein ‘n’ is the cycle number, ‘C₀’ is the initial target load and ‘b’ is a base number that is, in methods of the invention greater than 2 and that is commonly referred to herein as “amplification power” or “amplification power coefficient.” The amplification power coefficient can be determined by a method that is well established in the art and that is based on target load titration as illustrated herein in FIGS. 5B-8B. The amplification power coefficient ‘b’ was calculated according to equation b=10^{(1/-s)} using the slope value ‘s’ from the linear trend equation (see FIGS. 5B-8B). In this aspect, the amplification power coefficients determined herein represent an average value throughout/over most or all PCR cycles.

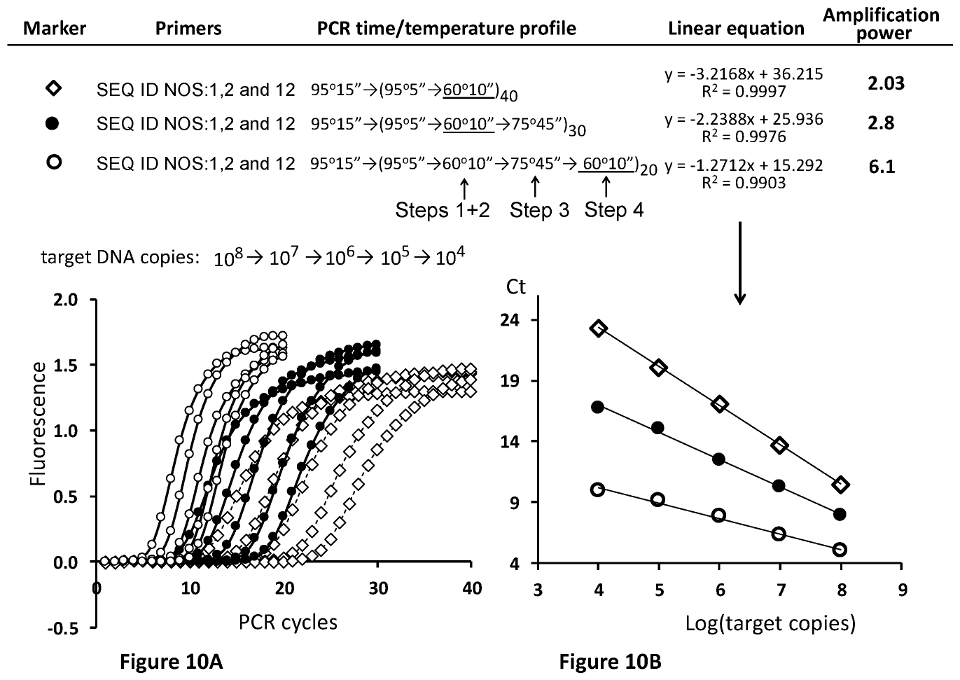

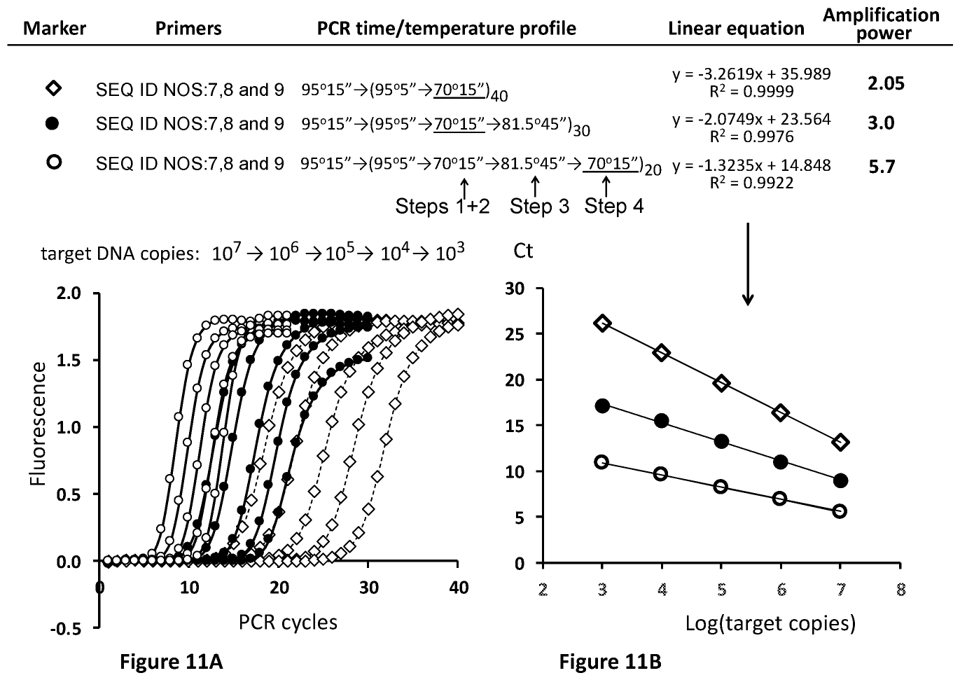

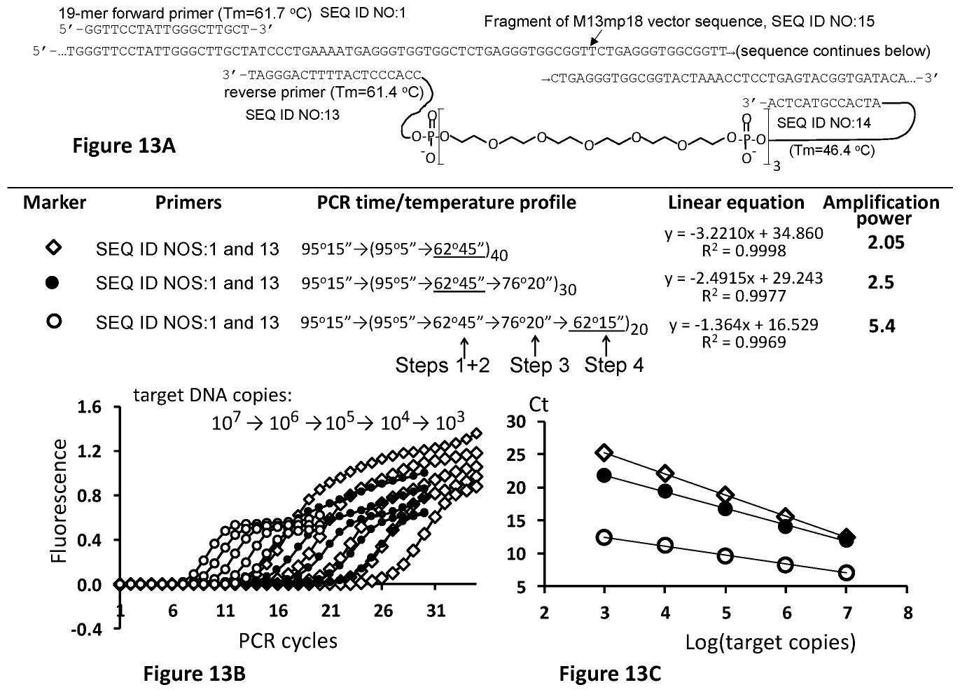

In exemplary three-primer embodiments, methods of the invention are based on use of three oligonucleotide primers (P1, P2 and P3) as illustrated in FIGS. 1A-1C. In exemplary four-primer embodiments, methods of the invention are based on use of four oligonucleotide primers (P1, P2, P3, and P4) as illustrated in FIGS. 2A and 2B. The terms “oligonucleotide primer” and/or “primer” refer to a single-stranded DNA or RNA molecule that hybridizes to a target nucleic acid and primes enzymatic synthesis of a complementary nucleic acid strand in presence of a DNA polymerase. In this case, as used herein, the target nucleic acid “serves as a template” for the oligonucleotide primer. As used herein, “hybridizing the third oligonucleotide primer to the target sequence at a position 5′ upstream from the hybridized second primer extension product,” means that there is a gap of at least one or more nucleotides of the target sequence between the primers' binding sites, e.g. as illustrated in FIGS. 1A-1C. In the methods, primers of the invention may hybridize immediately adjacent to each other without a nucleotide gap, as in the experiment of FIGS. 10A-B. Moreover, in the methods, the primers may overlap by one, two or more nucleotides, i.e., when binding site of one primer incorporates nucleotides of the binding site of another primer. Such binding site overlaps can be partial or complete, e.g., when the binding site sequence of the P3 primer incorporates the binding site of the P2 primer as illustrated, e.g. in the reactions of FIGS. 11A-B. In the methods, the P2 and P3 primers can have the same binding site sequence, but still provide for acceleration. In this case, the P3 primer may form a more stable duplex with a target sequence than does the P2 primer. For example, the P3 primer may incorporate polymerase-compatible duplex-stabilizing modifications, whereas the P2 primer may comprise natural nucleotides or incorporate polymerase-compatible duplex-destabilizing modifications. In the methods, P2 and P3 primers may be covalently coupled to each other. The coupling positions may be anywhere within the primer sequences as long as the coupling does not preclude the ability of these primers to perform in PCR. Perhaps the most convenient way to couple P2 and P3 primers is through use of a linker, as illustrated in FIG. 13A. The primers-coupled linker can be short, e.g., comprising one or more carbon atoms and/or phosphodiester moieties connecting the 5′-hydroxy groups of the P2 and P3 primers.

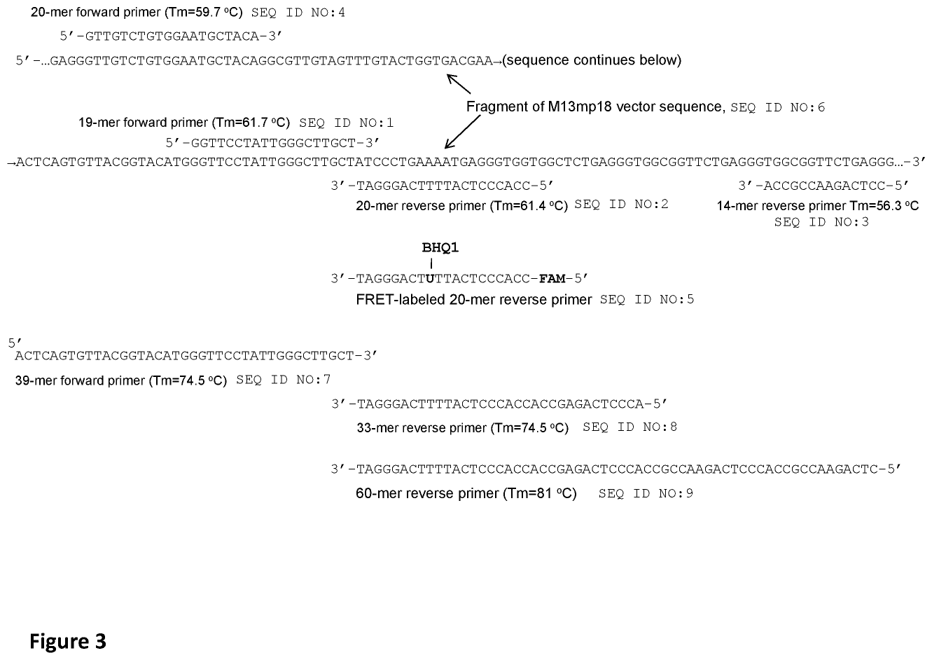

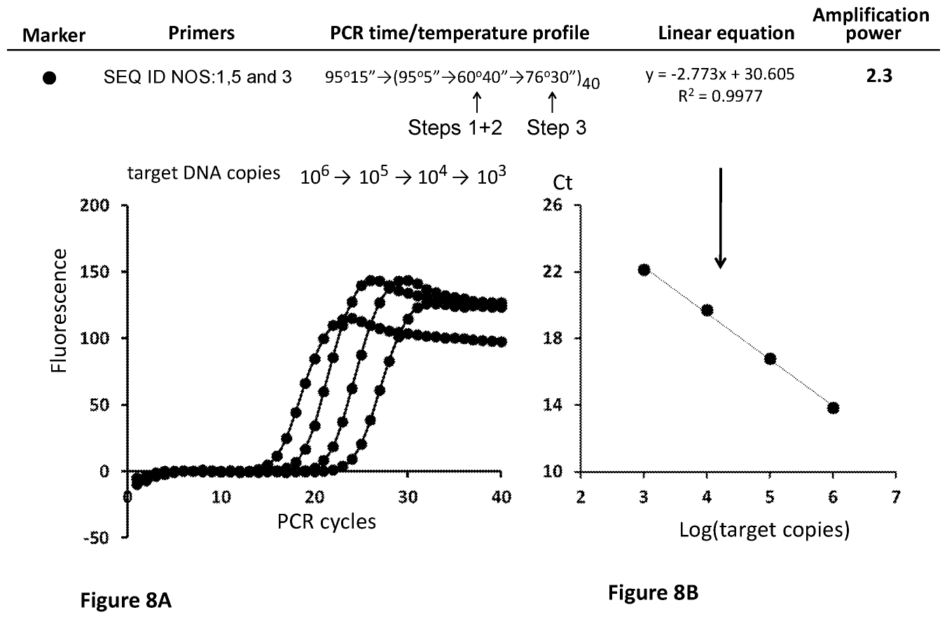

The term an “oligonucleotide probe” or “probe” refers to an oligonucleotide component which is used to detect nucleic acids of interest. These terms encompass various derivative forms such as “hybridization-triggered probe,” “fluorescent probe,” “FRET probe,” etc. Oligonucleotides can serve more than one function in PCR, for example, in methods of the invention an oligonucleotide can be a primer that provides for amplification of a target nucleic acid and it also can serve for the real time detection (i.e. usually a function of a “probe”) when it is appropriately labeled by FRET dyes (e.g., FIG. 3, SEQ ID NO:5) as exemplified in FIG. 8.

In the methods, the phrase “incubating the reaction mixture at a temperature sufficient to initiate thermal melting,” as used herein, means an exposure of the reaction mixture to a temperature or temperature range at which a “desired effect,” i.e. initiation of thermal melting of duplexes formed by P2 primer extension products in the exemplary methods of FIG. 1B, and the extension products of primers P1 and P2 in the exemplary methods of FIG. 2B. In methods of the invention, the P3 and/or P4 primer extension products are designed/selected in relation to the target sequence to retain an adequate thermal stability at the “sufficient temperature” to support their further extension when the P1 and/or P2 primer extension products are melted, and this is an important factor that determines the sufficient temperature. For example, the lower limit of the sufficient temperature range may be determined by the thermal stabilities of the P1 and/or P2 of extension products, whereas the upper limit of the sufficient temperature range may be controlled by the thermal stabilities of the P3 and/or P4 of extension products. In this manner, therefore, the term “sufficient temperature” incorporates the term “sufficient temperature range”. The greater the difference in thermal stabilities between the P1/P2 extension products and the P3/P4 extension products, the broader the sufficient temperature range at which the desired effect can be reached. In the methods, the reaction mixture may be incubated at a particular “sufficient temperature.” Alternatively, in the methods, the temperature may change or fluctuate during the incubation between the lower and upper limits of the “sufficient temperature range.” In the methods, the time of the incubation at the sufficient temperature will depend on the sufficient temperature applied. The closer the sufficient temperature applied is to the lower limit of the sufficient temperature range, the longer the incubation time it will take to reach the desired effect discussed above. In the methods, for example, the sufficient temperature may be selected depending on the desired incubation time at that sufficient temperature. For example, the sufficient temperature or range thereof may be selected so that the desired effect, e.g., an amplification power greater than 2, can be reached in a short time like, e.g., 1 second. When primers are designed according to the parameters or rules of the invention discussed herein, a modest but still detectable PCR acceleration can be achieved during a short exposure of the reaction mixture to a sufficient temperature range during the instrument heat-ramping, e.g. as illustrated in FIGS. 10A-B, 11A-B and 13A-B (data labeled by empty diamond symbols (0). In methods of the invention, depending on the sufficient temperature or range thereof, the time of exposure of the reaction mixture at the sufficient temperature may be, for example, 1 second or longer, preferably 2, 3, 4 seconds, or longer and more preferably 5, 6, 7, 8, 9, or 10 seconds or longer, or 30 seconds or longer. The longer the time of exposure of the reaction mixture at the sufficient temperature or temperature range, the better the level of completion of steps 3A and 3B in FIGS. 1B, 2B, 9 and 12 and the greater the achievable amplification power of the PCR. In the methods, primer design (e.g., nucleotide sequence and relative spatial positioning of the primer binding sites in relation to the target sequence) may be used to configure the accelerated PCR methods with particular desired sufficient temperatures or sufficient temperature ranges, and incubation times at those desired sufficient temperatures or at those sufficient temperature ranges. In the methods, the sufficient temperatures or sufficient temperature ranges, and incubation times at those desired sufficient temperatures or at those sufficient temperature ranges are preferably selected to increase the amplification power to a value greater than 2, greater than 2.2, greater than 2.5, greater than 5, greater than 5.5, greater than 6, greater than 6.4, etc., to provide the greatest accelerated PCR.

In methods of the invention, “sample” refers to any substance containing or presumed to contain a nucleic acid of interest. The term “sample” thus includes but is not limited to a sample of nucleic acid, cell, organism, tissue, fluid, or substance including but not limited to, for example, blood, plasma, serum, urine, tears, stool, respiratory and genitourinary tracts, saliva, semen, fragments of different organs, tissue, blood cells, samples of in vitro cell cultures, isolates from natural sources such as drinking water, microbial specimens, and objects or specimens that have been suspected to contain nucleic acid molecules.

In methods of the invention, the term “reaction mixture” generally means an aqueous solution comprising all the necessary reactants including oligonucleotide components, enzymes, nucleoside triphosphates (dNTPs), ions like magnesium and other reaction components for performing an amplification or detection reaction of the invention or both. Magnesium ion is preferably present in the reaction mixture because it enables catalytic activity of DNA polymerases. Additional, non-necessary components may be included in the reaction mixture, as long as they don't preclude the methods.

In methods of the invention, “polynucleotide” and “oligonucleotide” are used herein interchangeably and each means a linear polymer of nucleotide monomers. Polynucleotides typically range in size from a few monomeric units, e.g., 5-40, when they are usually referred to as “oligonucleotides,” to several thousand monomeric units. The exact size will depend on many factors, which in turn depends on the ultimate function or use of the oligonucleotide. The oligonucleotides may be generated in any manner, including chemical synthesis, DNA replication, reverse transcription, or a combination thereof. Whenever a polynucleotide or oligonucleotide is represented by a sequence of letters, for example, “CCGTATG,” it is understood herein, unless otherwise specified in the text, that the nucleotides are in 5′ to 3′ forward order from left to right and that “A” denotes deoxyadenosine, “C” denotes deoxycytidine, “G” denotes deoxyguanosine, and “T” denotes deoxythymidine. Usually DNA polynucleotides comprise these four deoxyribonucleosides linked by phosphodiester linkage whereas RNA comprises uridine (“U”) in place of “T” for the ribose counterparts.

As used herein, the term “producing a primer extension product” describes two steps of the primer-assisted DNA synthesis such as (i) hybridization of a primer to a target sequence strand and then (ii) extension of this primer by DNA polymerase in presence of deoxynucleoside 5′-triphosphates. In methods of the invention, “hybridizing,” “hybridization,” or “annealing” refers to a process of interaction between two or more oligo- and polynucleotides forming a complementary complex through base pairing which is most commonly a duplex. The stability of a nucleic acid duplex is measured by its melting temperature. “Melting temperature” or “Tm” means the temperature at which a complementary duplex of nucleic acids, usually double-stranded, becomes half dissociated into single strands. These terms are also used in describing stabilities of secondary structures wherein two or more fragments or portions of the same polynucleotide interact in a complementary fashion with each other forming duplexes (e.g., hairpin-like structures). “Hybridization properties” of a polynucleotide means an ability of this polynucleotide or a fragment or portion thereof to form a sequence specific duplex with another complementary polynucleotide or a fragment or portion thereof. The term “hybridization properties” is also used herein as a general term in describing a complementary duplex stability. In this aspect, “hybridization properties” are similar in use to “melting temperature” or “Tm.” “Improved” or “enhanced hybridization properties” of a polynucleotide refers to an increase in stability of a duplex of this polynucleotide with its complementary sequence due to any means including but not limited to a change in reaction conditions such as pH, salt concentration, and composition, for example, an increase in magnesium ion concentration, presence of duplex stabilizing agents such as intercalators or minor groove binders, etc., conjugated or not. The hybridization properties of a polynucleotide or oligonucleotide can also be altered by an increase or decrease in polynucleotide or oligonucleotide length. The cause of the hybridization property enhancement or detraction is generally defined herein in context. A simple estimate of the Tm value can be made using the base pair thermodynamics of a “nearest-neighbors” approach (Breslauer, K. J., et al., 1986; SantaLucia, J., Jr., 1998). Commercial programs, including Oligo™, Primer Design and programs available on the internet like Primer3™, and Oligo Calculator™, can be also used to calculate a Tm of a nucleic acid sequence useful according to the invention. Commercial programs, e.g., Visual OMP™, (DNA software), Beacon designer 7.00™. (Premier Biosoft International), may also be helpful.

In methods of the invention, the term “structural modifications” refers to any chemical substances such as atoms, moieties, residues, polymers, linkers or nucleotide analogs that are usually of a synthetic nature, and which are not commonly present in natural nucleic acids. “Duplex-stabilizing modifications” refer to structural modifications, the presence of which provide a duplex-stabilizing effect in double-stranded nucleic acids; that is such modifications enhance thermal stability (e.g., “Tm”) relative to nucleic acid duplexes lacking such stabilizing modification(s) (e.g., that contain only natural nucleotides). Conversely, “duplex-destabilizing modifications” refer to structural modifications, the presence of which provide a duplex-destabilizing effect (e.g., decreased thermal stability/Tm) in double-stranded nucleic acids. Duplex-stabilizing modifications include those structural modifications that are most commonly applied in synthesis of probes and primers and are represented by modified nucleotides and “tails” and may include intercalators and minor groove binders. Particularly useful in methods of the invention are “polymerase-compatible” structural modifications incorporated into the oligonucleotide primers.

The “polymerase-compatible” structural modifications refer to modifications that do not block DNA polymerase activity in extending the hybridized primers and/or that replicate the primer sequence incorporating these modifications. Use of polymerase-efficient modifications in primer design can be beneficial in methods of the invention. For example, the P3 and P4 primers used in exemplary methods described herein may incorporate polymerase-compatible duplex-stabilizing modifications to stabilize their primer extension products at the “sufficient temperature” as defined herein above. Similarly, the P1 and P2 primers used in exemplary methods described herein may incorporate polymerase-compatible duplex-destabilizing modifications to destabilize their primer extension products at step 3 of the exemplary methods (e.g., see step 3 in the schemes of FIG. 1B or 2B, wherein these primer extension products are rendered single-stranded). Examples of polymerase-compatible duplex-stabilizing modifications include but are not limited to 5′-conjugated intercalators (e.g., Lokhov, S. G., et al. (1992), minor groove binding moieties (e.g., Hedgpeth, J., et al., (2010) U.S. Pat. No. 7,794,945), 5-methyl cytosine (5-MeC) and 2,6-diamino-purine (2-amA) nucleotide analog in place of cytosine and adenine, respectively (e.g., Lebedev, Y., et al., 1996). Examples of polymerase-compatible duplex-destabilizing modifications include but are not limited to 7-deaza purine nucleotide analogs (Seela, et al., 1992), deoxyinosine and deoxyuridine nucleotides (Kawase, Y., et al., 1986, Martin, F. H., et al., 1985).

In the methods, the terms “natural nucleosides” and “natural nucleotides” as used herein refer to four deoxynucleosides or deoxynucleotides respectively which may be commonly found in DNAs isolated from natural sources. Natural nucleosides (nucleotides) are deoxyadenosine, deoxycytidine, deoxyguanosine, and deoxythymidine. The term also encompasses their ribose counterparts, with uridine in place of thymidine. As used herein, the terms “unnatural nucleotides” or “modified nucleotides” refer to nucleotide analogs that are different in their structure from those natural nucleotides for DNA and RNA polymers. Some of the naturally occurring nucleic acids of interest may contain nucleotides that are structurally different from the natural nucleotides defined above, for example, DNAs of eukaryotes may incorporate 5-methyl-cytosine and tRNAs are notorious for harboring many nucleotide analogs. However, as used herein, the terms “unnatural nucleotides” or “modified nucleotides” encompasses these nucleotide modifications even though they can be found in natural sources. For example, ribothymidine and deoxyuridine are treated herein as unnatural nucleosides. In this aspect, the discussed above deoxyinosine and deoxyuridine nucleosides are unnatural nucleosides.

In methods of the invention, the terms “complementary” or “complementarity” are used herein in reference to the polynucleotides base-pairing rules. Double-stranded DNA, for example, consists of base pairs wherein, for example, G forms a three hydrogen bonds, or pairs with C, and A forms a two hydrogen bonds complex, or pairs with T, and it is regarded that G is complementary to C, and A is complementary to T. In this sense, for example, an oligonucleotide 5′-GATTTC-3′ is complementary to the sequence 3′-CTAAAG-5′. Complementarity may be “partial” or “complete.” In partial complementarity, only some of the nucleic acids' bases are matched according to the base pairing rules. The degree of complementarity between nucleic acid strands has significant effects on the strength of hybridization between nucleic acids. This is particularly important in performing amplification and detection reactions that depend upon nucleic acid binding interactions. The terms may also be used in reference to individual nucleotides and oligonucleotide sequences within the context of polynucleotides. As used herein, the terms “complementary” or “complementarity” generally refer to the most common type of complementarity in nucleic acids, namely, Watson-Crick base pairing as described above, although the primers, probes and amplification products of the invention may also participate, including in intelligent design, in other types of “non-canonical” pairings like Hoogsteen, wobble and G-T mismatch pairing.

In methods of the invention, the term “design” in the context of the method steps and/or oligonucleotides, etc., has broad meaning, and in certain aspects is equivalent to the term “selection.” For example, the terms “oligonucleotide design,” “primer design,” “probe design” can mean or encompass selection of a type, a class, or one or more particular oligonucleotide structure(s) including the nucleotide sequence and/or structural modifications (e.g., labels, modified nucleotides, linkers, etc.). The term “system design” generally incorporates the terms “oligonucleotide design,” “primer design,” “probe design” and also refers to relative orientation and/or location of the oligonucleotide components and/or their binding sites within the target nucleic acids. In these aspects, the term “assay design” relates to the selection of any, sometimes not necessarily to a particular, methods including all reaction conditions (e.g. temperature, salt, pH, enzymes, oligonucleotide component concentrations, etc.), structural parameters (e.g. length and position of primers and probes, design of specialty sequences, etc.) and assay derivative forms (e.g. post-amplification, real-time, immobilized, FRET detection schemes, etc.) chosen to amplify and/or to detect the nucleic acids of interest.

In methods of the invention, “detection assay” or “assay” refers a reaction or chain of reactions that are performed to detect nucleic acids of interest. The assay may comprise multiple stages including amplification and detection reactions performed consecutively or in real-time, nucleic acid isolation and intermediate purification stages, immobilization, labeling, etc. The terms “detection assay” or “assay” encompass a variety of derivative forms of the methods of the invention, including but not limited to, a “post-amplification assay” when the detection is performed after the amplification stage, a “real-time assay” when the amplification and detection are performed simultaneously, a “FRET assay” when the detection is based using a FRET effect, “immobilized assay” when one of either amplification or detection oligonucleotide components or an amplification product is immobilized on solid support, and the like.

Methods of the invention can be used to amplify and detect one, or a plurality (more than one) of target nucleic acids in, for example, a multiplex detection format. The term “multiplexed detection” refers to a detection reaction wherein multiple or plurality of target nucleic acids are simultaneously detected. “Multiplexed amplification” correspondingly refers to an amplification reaction wherein multiple target nucleic acids are simultaneously amplified in the same reaction mixture.

In methods of the invention, products of the target amplification can be detected by any appropriate physical, chemical or biochemical approach. In preferred embodiments, the PCR amplification products comprise a detectable label. The term “label” refers to any atom or molecule that can be used to provide a detectable signal and that can be attached to a nucleic acid or oligonucleotide. Labels include but are not limited to isotopes, radiolabels such as ³²P, binding moieties such as biotin, haptens, mass tags, phosphorescent or fluorescent moieties, fluorescent dyes alone or in combination with other dyes or moieties that can suppress or shift emission spectra by FRET effects. Labels may provide signals detectable by fluorescence, radioactivity, colorimetry, gravimetry, X-ray diffraction or absorption, magnetism, enzymatic activity, mass spectrometry, binding affinity and the like. A label may be a charged moiety or alternatively, may be charge neutral. Labels can include or consist of nucleic acid or protein sequences, so long as the sequence comprising the label is detectable. In preferred embodiments, the label is a fluorescent label. “Fluorescent label” refers to a label that provides a fluorescent signal. A fluorescent label is commonly a fluorescent dye, but it may be any molecule including but not limited to a macromolecule like a protein, a particle made from inorganic material like quantum dots, as described, for example, in (Robelek, R., et al., 2004), etc.

In methods of the invention, the probes may be FRET probes and the detection of target nucleic acids may be based on FRET effects. “FRET” is an abbreviation of Forster Resonance Energy Transfer effect. FRET is a distance-dependent interaction occurring between two dye molecules in which excitation is transferred from a donor to an acceptor fluorophore through dipole-dipole interaction without the emission of a photon. As a result, the donor molecule fluorescence is quenched, and the acceptor molecule becomes excited. The efficiency of FRET depends on spectral properties, relative orientation and distance between the donor and acceptor chromophores (Forster, T., 1965). As used herein, “FRET probe” or “FRET primer” refers to a fluorescent oligonucleotide that is used for detection of a nucleic acid of interest, wherein detection is based on FRET effects. The acceptor chromophore may be a non-fluorescent dye chosen to quench fluorescence of the reporting fluorophore (Eftink, M. R., 1991). Formation of sequence-specific hybrids between the target nucleic acid and the probes or primer leads to changes in fluorescent properties providing for detection of the nucleic acid target. FRET is widely used in biomedical research and particularly in probe designs for nucleic acid detection (e.g., in Didenko, V. V., 2001).

Many detection strategies and designs exploiting the FRET effect are known in the art, and these strategies may be used in design of the FRET-labeled probes or FRET-labeled primers of the invention. In particular aspects, the FRET probes or FRET primers are hybridization-triggered FRET oligonucleotide components. The “hybridization-triggered” FRET approach is based on distance change between the donor and acceptor dyes as result of a sequence specific duplex formation between a target nucleic acid and a fluorescent oligonucleotide component. When a FRET-labeled oligonucleotide component is unhybridized, the quencher moiety is sufficiently close to the reporter dye to quench its fluorescence due to random oligonucleotide coiling. Once the FRET-labeled oligonucleotide component is hybridized to the primer-extension products forming rigid duplex, the quencher and reporter moieties are separated, thus enabling the reporter dye to fluoresce providing for the target nucleic acid detection (e.g., Livak, K. J., et al., 1998). Examples of other hybridization-triggered FRET system designs that can be used practicing the present invention include but not limited to “Adjacent Hybridization Probe” method (e.g., Eftink, M. R., 1991; Heller, M. J. and Morrison, L. E., 1985), “Molecular Beacons” (Lizardi, P. M., et al., 1992), “Eclipse Probes” (Afonina, I. A., et al., 2002), all of which are incorporated herein by reference for their relevant teachings. The exemplary experimental results shown of FIG. 8 and discussed in working Example 4 illustrate an embodiment of the invention wherein a FRET-labeled P2 primer (SEQ ID NO:5, FIG. 3) was used for the detection of amplified material. Other approaches that are based on use of FRET-labeled primers and that can be used in methods of the invention include those described, for example, in U.S. Pat. No. 9,353,405, issued to Rabbani, E., et al.; U.S. Pat. No. 5,866,336, issued to Nazarenko, I. A., et al.; U.S. Patent Application Publication No. 2012/0058481, by Ge, W., et al., all of which are incorporated herein by the reference.

In methods of the invention, the amplification and detection stages of the invention may be performed separately when the detection stage follows the amplification. The terms “detection performed after the amplification,” “target nucleic acid is amplified before the detection reaction” and “post-amplification detection” are used herein to describe such assays. In preferred method embodiments of the invention, detection of target nucleic acids can be performed in “real-time.” Real-time detection is possible when all detection components are available during the amplification, and the reaction conditions (e.g., temperature, buffering agents to maintain pH at a selected level, salts, co-factors, scavengers, and the like) support both amplification and detection stages of the reaction. This permits a target nucleic acid to be measured as the amplification reaction progresses, decreasing the number of subsequent handling steps required for the detection of amplified material. “Real-time detection” means an amplification reaction for which the amount of reaction product, (e.g., target nucleic acid sequences), is monitored as the reaction proceeds. Reviews of the detection chemistries for real-time amplification can be found, for example, in Didenko, V. V., 2001, Mackay, I. M., et al., 2002, and Mackay, J., and Landt, O., 2007, which are incorporated herein by reference for their relevant teachings. In preferred embodiments of the present invention, real-time detection of nucleic acids is based on use of FRET effect, FRET-labeled probes or primers. In certain aspects, detection of amplified nucleic acid material can be performed using certain technologies based on nuclease-cleavable probes. Examples include but are not limited to chimeric DNA-RNA probes that are cleaved by RNAse H upon the binding to target DNA (see, e.g., Duck, P., et al., 1989); target-specific probe cleavage based on the substrate specificity of Endonuclease IV and Endonuclease V from E. coli (Kutyavin, I. V., et al., 2007).

The reaction components to perform methods of the invention can be delivered in the form of a kit. As used herein, the term “kit” refers to any system for delivering materials. In the context of PCR methods/reaction assays, such delivery systems include elements allowing the storage, transport, or delivery of reaction components such as oligonucleotides, buffering components, additives, reaction enhancers, enzymes and the like in the appropriate containers from one location to another commonly provided with written instructions for performing the assay. Kits may include one or more enclosures or boxes containing the relevant reaction reagents and supporting materials. The kit may comprise two or more separate containers wherein each of those containers includes a portion of the total kit components. The containers may be delivered to the intended recipient together or separately.

The oligonucleotide components of the invention such as primers and probes can be prepared by a suitable chemical synthesis method. The preferred approach is the diethylphosphoramidate method disclosed in Beaucage, S. L., Caruthers, M. H. (1981), in combination with the solid support method disclosed in Caruthers, M. H., Matteucci, M. D. (1984), and performed using one of commercial automated oligonucleotide synthesizer. When oligonucleotide components of the invention, primers or probes, need to be labeled with a fluorescent dye a wide range of fluorophores may be applied in designs and synthesis. Available fluorophores include but not limited to coumarin, fluorescein (FAM, usually 6-fluorescein or 6-FAM), tetrachlorofluorescein (TET), hexachloro fluorescein (HEX), rhodamine, tetramethyl rhodamine, BODIPY, Cy3, Cy5, Cy7, Texas red and ROX. Fluorophores may be chosen to absorb and emit in the visible spectrum or outside the visible spectrum, such as in the ultraviolet or infrared ranges. FRET probes or primers of the invention commonly incorporate a pair of fluorophores, one of which may be a none-fluorescent chromophore (commonly referred as a “dark quencher”). Suitable dark quenchers described in the art include Dabcyl and its derivatives like Methyl Red. Commercial non-fluorescent quenchers, e.g., Eclipse™ (Glen Research) and BHQ1, BHQ2, BHQ3 (Biosearch Technologies), may be also used for synthesis of FRET primers and probes of the invention. Preferred quenchers are either dark quenchers or fluorophores that do not fluoresce in the chosen detection range of the assays. Modified nucleoside or nucleotide analogs, for example, 5-methyl cytosine, 2-amino adenosine (2,6-diaminopurine), deoxyinosine and deoxyuridine, which are rarely present in natural nucleic acids may be incorporated synthetically into oligonucleotide components. The same applies to linkers, spacers, specialty tails like intercalators and minor groove binders. All these chemical components can be prepared according to methods of organic chemistry or using respective protocols that can be found in manuscripts and patents cited herein. Many structural modifications and modified nucleosides useful to prepare oligonucleotide components of the invention are available, commonly in convenient forms of phosphoramidites and specially controlled pore glass, from commercial sources, e.g., Glen Research, Biosearch Technologies, etc.

DNA polymerases are key components in practicing amplification and detection assays of the invention. DNA polymerases useful according to the invention may be native polymerases as well as polymerase mutants, which are commonly modified to improve certain performance characteristics or to eliminate 5′ to 3′ and/or 3′ to 5′ exo nuclease or endo nuclease activities that may be found in many native enzymes. Nucleic acid polymerases can possess different degrees of thermostability. Preferably, for performing the PCR methods of the invention, DNA polymerases are stable at temperatures >90° C., preferably >95° C., and even more preferably >100° C. Preferably the DNA polymerases have no 5′-3′ exonuclease activity found, for example, in Taq polymerase. Examples of thermostable DNA polymerases which are useful for performing the PCR methods of the invention include but are not limited to Vent, Vent(exo-), Deep Vent, Deep Vent(exo-) (New England Biolabs), SD polymerase (Bioron GmbH), Top polymerase (Bioneer) and other polymerase from Thermus species. The presence or absence in DNA polymerases of the 3′ to 5′ nuclease activity, which is known in the art as “proofreading” nuclease activity, is not as significant for many methods of the invention as other characteristics such as the enzyme processivity, strand displacing activity, affinity to primer-extension complex and DNA synthesis speed. The DNA polymerases used in methods of the invention preferably have no associated nuclease activity. An example of such a DNA polymerase is Top polymerase (Bioneer) successfully used in the exemplary methods provided herein (FIGS. 5-8).

Nucleic acids of interest are commonly present in test samples at a low concentration which does not allow for direct detection. Amplification of the target nucleic acids is needed, and PCR is the most common choice of the amplification technique, although other technologies, e.g., isothermal amplification schemes, are emerging. PCR may amplify nucleic acids to a nanomolar range of concentrations starting from as little as a single molecule of the nucleic acid of interest. Nanomolar concentrations are well within the detection range of fluorescence-based technologies, providing a convenient way for detection of the amplification products, particularly in real time.

### Exemplary Three-Primer Accelerated PCR Embodiment

With reference to FIGS. 1A-1C, FIG. 1A illustrates, according to particular exemplary aspects of the present invention, amplifying a target nucleic acid sequence (e.g., DNA) in a sample using three oligonucleotide primers P1, P2, and P3 during a first PCR cycle. Target nucleic acid sequences in the sample may initially be present in long polymeric molecules. For convenience of illustration, the exemplary target strands (shown at the top left corner of FIG. 1A) that are amplified during PCR are shown in solid lines, whereas adjacent contiguous sequences are shown by dashed lines. In stage A, primers P1 and P2 hybridize to complementary primer binding sites on corresponding first and second target strands, respectively, and are extended in the presence of a DNA polymerase and other reaction components necessary for DNA synthesis. In stage B, primer P3 hybridizes to a complementary primer binding site on the second strand of the target sequence at a position 5′ upstream from the hybridized primer P2 and is extended toward (e.g., up to) the 5′-end of the P2 primer extension product, which in this instance may be long and highly thermostable due to its indefinite 3′-end. As explained below, initiation of melting of this particular initial P2 extension product does not occur at a “sufficient temperature” as defined herein, and thus the initial thermostable P2 extension product precludes the P3 primer extension product from extending beyond the 5′-end of the P2 primer extension product. If the P3 primer extension product could efficiently extend further during this first cycle and subsequent cycles of the PCR (e.g., up to a position incorporating the P1 primer binding site), then it would be beneficial for the methods of the invention. FIG. 1A illustrates a worst-case scenario when the initial P2 primer extension product completely blocks further extension of the initial P3 primer extension product. This blocked initial P3 primer extension product does not incorporate a primer P1 binding site (shown as an “X” at its 3′-end, FIG. 1A), and thus is effectively wasted because it cannot participate in further amplification during subsequent PCR cycles. As shown in stage C, denaturation of all primer extension products by incubating the reaction mixture at a denaturation temperature, recovers the first and second target strands with indefinite ends and produces P1 and P2 primer extension products having indefinite ends. According to particular aspects of the present invention, formation of these two single-stranded products during denaturation after the first cycle of PCR provides for accelerated PCR in a next, successive cycle of the PCR, as illustrated in FIG. 1B. In this aspect, accelerated PCR, as described in detail below, may not take place during a first PCR cycle, e.g., where the initial primer extension products have indefinite 3′-ends, but the first PCR cycle nonetheless produces two key P1 and P2 primer extension products that provide for accelerated PCR in consecutive cycles.

FIG. 1B illustrates, according to particular exemplary aspects of the present invention, amplifying the target nucleic acid sequence using the three oligonucleotide primers P1, P2, and P3 during a next, successive cycle of the PCR (second PCR cycle). In step 1, additional primers P1 and P2 hybridize to complementary sites on the P2 and P1 primer extension products, respectively, produced in the first PCR cycle shown in FIG. 1A, and are extended to form respective hybridized extension products. In step 2, an additional primer P3 also hybridizes to the P1 primer extension product produced during first PCR cycle and is extended up to the 5′-end of the P2 primer extension product that is hybridized downstream. According to particular aspects of the present invention, the reaction mixture is then incubated at a temperature (referred to herein as the “sufficient temperature”) lower than the denaturation temperature, but sufficient to initiate thermal melting of the hybridized P2 primer extension product (step 3A), and where the hybridized P3 primer extension product has a thermal stability sufficient to provide for its further extension at the sufficient temperature (step 3B) to produce a double-stranded, further extended P3 primer extension product incorporating a P1 primer binding site at its 3′ end. According to additional aspects of the present invention, initiation of thermal melting of the hybridized P2 primer extension product at the ‘sufficient’ temperature and further extension of the P3 product is accompanied by provision of the P2 primer extension product in a single stranded form at the end of step 3B. According to further aspects of the present invention, the P1 primer extension product hybridized to the P2 primer extension product produced in the first PCR cycle has nearly the same thermal stability as the P2 primer extension product hybridized to the P1 primer extension product produced at step 1 and is therefore also melted at the ‘sufficient’ temperature.

In particular exemplary aspects of the present invention, the PCR cycle and amplification can be terminated at the completion of step 3B by increasing the reaction temperature to the denaturation temperature, where all hybridized primer extension products are denatured-effectively bypassing step 4. Experiments described herein, and the results shown in FIGS. 5, 6, and 8 (curves and data labeled by black dots) indicate that this truncated method nonetheless leads to appreciable acceleration of PCR (accelerated PCR; e.g., amplification power of 2.5).

In preferred exemplary aspects, in order to further increase the PCR amplification power, the reaction temperature is lowered in step 4 of this next successive cycle of the PCR, to a temperature below the sufficient temperature to enable hybridization of primers P1 and P2 to the single-stranded extension products of these primers produced in step 3. Subsequent extension of these complexes by DNA polymerase results in double stranded amplification products incorporating P1 and P2 primer binding sites at their 3′ ends. According to yet further aspects of the present invention, when step 4 is included, the P1 primer extension product is amplified twice (once prior to incubating the reaction mixture at the sufficient temperature, and once thereafter) during this next, successive cycle of the PCR. Upon completion of step 4, the reaction temperature is raised to the denaturation temperature to denature all double stranded amplification products rendering them single stranded for a further successive cycle of the PCR. According to additional aspects of the present invention, the original single-stranded first and second target strands with indefinite ends participate in amplification during the next successive cycle, as well as all other consecutive cycles, each time producing P1 and P2 primer extension products according to the scheme of FIG. 1A.

FIG. 1C shows the amplification products present in reaction mixture at the beginning (left) and at the end (right) of further successive cycle of the PCR (third cycle) incorporating all four steps (step 1 through step 4). According to the reaction scheme of FIGS. 1A-1C), the amounts of the long amplicons framed by the sequences of P1 and P3 primers is going to double in each consecutive PCR cycle following the traditional PCR fashion as practiced in the prior art. However, the number of the short amplicons framed by the sequences of P1 and P2 primers grows at a much faster rate. In particular, amplification of these short products strongly accelerates PCR, because many of them are replicated twice in each consecutive PCR cycle because of use of the sufficient temperature, lower than the denaturation temperature, to initiate melting of the P2 primer extension products (and of the P1 primer extension products having P2 primer binding sites at their 3′-ends). Moreover, the P2 primer extension on denatured strands of the long amplicons in subsequent cycles steadily supplies short amplicons into the PCR reaction, making it difficult to estimate the upper limit of amplification power in these exemplary methods of the invention.

As will be appreciated from the above description, therefore, in one truncated aspect of the three-primer embodiment of this invention, the reaction temperature may be raised to the denaturation temperature to denature the primer extension products once the reactions of steps 3A and 3B are accomplished (see steps 3A and 3B of FIG. 1B)—effectively by passing step 4. Preferred aspects of the three-primer embodiment, however, incorporate step 4, wherein another first oligonucleotide primer P1, present in excess relative to the target sequence, hybridizes to the second primer extension product not hybridized to the second strand of the target sequence, and is extended to provide a double-stranded extension product having first and second oligonucleotide primer binding sites at its 3′ ends.

According to particular aspects of the present invention, the hybridization of the first primer P1 may be thermally destabilized to some degree at the ‘sufficient’ temperature, and therefore its hybridization and extension in step 4 may be facilitated by lowering the reaction temperature after steps 3A and 3B (e.g., in the schemes of FIGS. 1B-C) as was performed in particular working Examples disclosed herein, the results of which is provided in FIGS. 5-8.

Both method aspects, without and with step 4 incorporated into the PCR, result in accelerated PCR, but at a different degree. As evident from the working Examples and the results shown in FIGS. 5-8, embodiments incorporating step 4 are the most powerful in terms of PCR acceleration, primarily due to the fact that at least many, or most, or substantially all of the short amplicons incorporating P1 and P2 binding sites at their 3′-ends are amplified twice during each cycle of the PCR. Although, according to the accelerated PCR scheme shown in FIGS. 1A-C, the amount of long amplicons incorporating P1 and P3 binding sites at their 3′-ends can only double in each consecutive cycle, similar to a conventional PCR, amplification of these long primer extension products is nonetheless an important acceleration-driving factor, because they provide a steady supply of short amplicons into the reaction system from cycle to cycle.

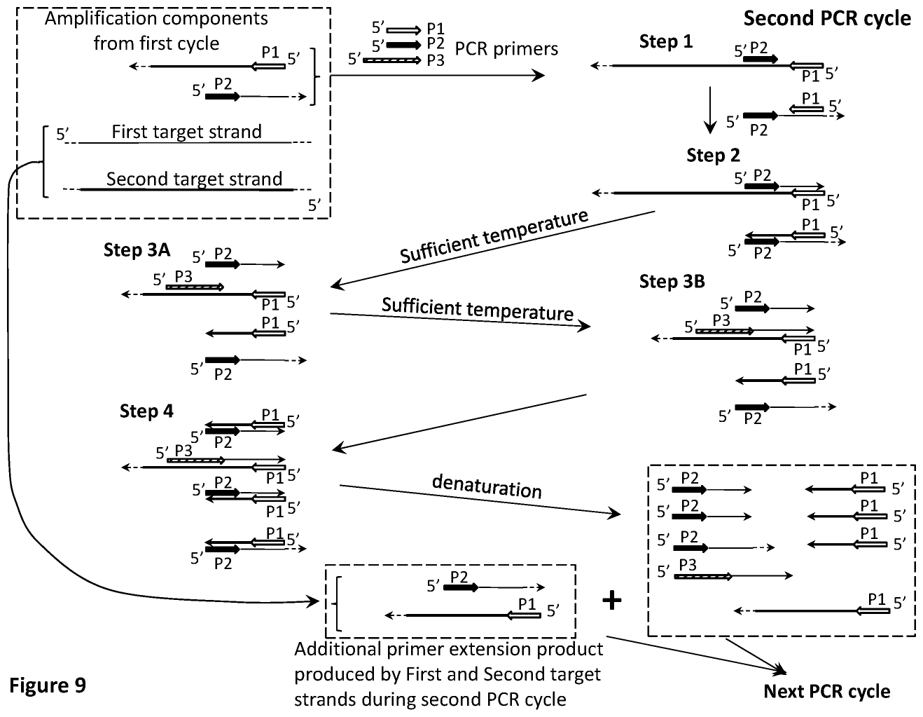

FIGS. 1A-1C illustrate a scenario wherein the P3 primer hybridizes to the target strand at a position 5′ upstream from the hybridized P2 primer (no overlap). The original P3 primer may have relatively low duplex stability but, after its extension up to the 5′-end of the P2 primer extension product, it gains in thermal stability to withstand the ‘sufficient’ temperature at which thermal melting of the P2 primer extension product is initiated. According to particular aspects of the present invention, the original P3 primer (i.e., without extension) may have adequate properties to hybridize to a target strand and then to be extended by a DNA polymerase at the ‘sufficient’ temperature. This is particularly relevant to embodiments wherein the P3 primer binding site overlaps the binding site of P2 primer as illustrated in FIG. 9, or wherein the P3 primer binding site is located immediately adjacent to 3′-end of the P2 primer binding site. Although the P3 primer may have greater hybridization stability than that of the P2 primer, the dominance in hybridization and extension of the P2 primer over that of the P3 primer during the instrumental cool-ramping can be achieved by application of a ‘kinetic’ factor as discussed below, e.g., when the P2 primer is used at a concentration that is greater than that of the P3 primer. As illustrated in FIGS. 10 and 11, ‘disbalancing’ of P2 and P3 primers' relative concentration provided for accelerating PCR to significant power levels of 2.8 and 3.0, respectively. Incorporation of the step 4 into the time/temperature profile further accelerates PCR resulting in amplification power of 6.1 and 5.7, respectively.

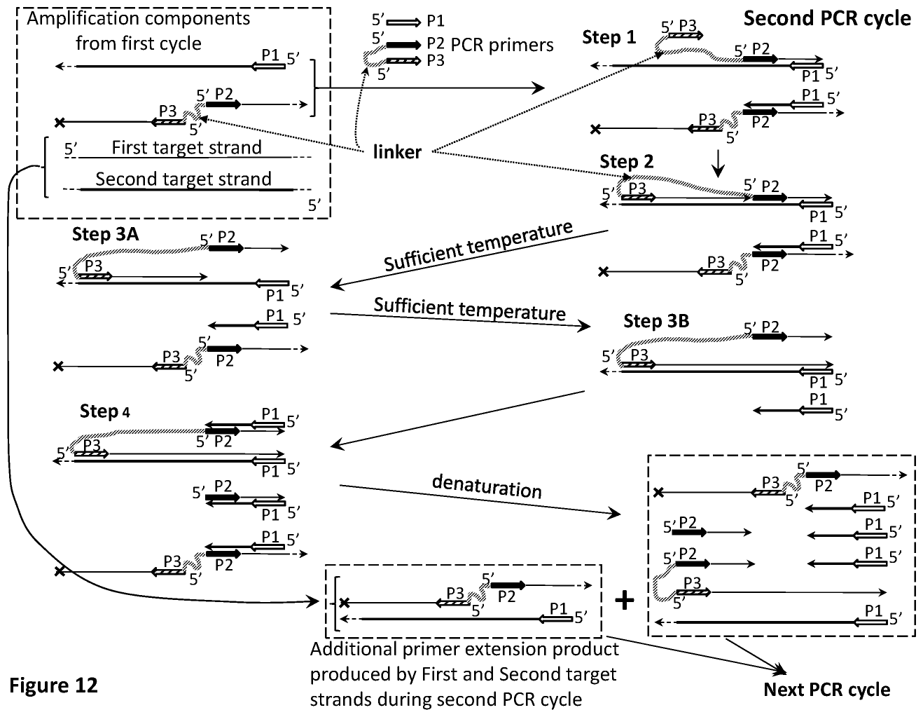

In the methods (e.g., the methods of FIGS. 1A-C), P2 and P3 primers may be covalently coupled to each other, e.g. by their 5′-ends through a linker, as illustrated in FIG. 12, which shows the amplification steps, including step 4, during a second PCR cycle (following a first PCR cycle, the products of which are shown in the dashed box in the upper left corner of FIG. 12). While the reaction diagram of the first cycle for this method embodiment is not shown, in analogy to the method embodiment of FIG. 1A, the P3 primer coupled to the P2 primer may hybridize to the second target strand during the first PCR cycle and prime DNA replication up to the 5′-end of P2 primer, and as shown in FIG. 12 this P3 primer extension product (shown with an “X” at its 3′-end) does not incorporate a P1 primer binding site (for the same reason as discussed above for FIG. 1A) and thus is effectively wasted because it cannot participate in further amplification during subsequent PCR cycles. The mechanism of action of the second and subsequent cycles of the method embodiment of FIG. 12 is similar to that of FIGS. 1B-C in many aspects. However, in the method of FIG. 12, the P3 primer may have lower duplex stability than the P2 primer, as illustrated, e.g., in FIG. 13A, but may still effectively perform at the hybridization and extension temperature of P2 primer. This is because, unlike in the method of FIGS. 1A-C, the targeted hybridization of the P3 primer coupled to the P2 primer becomes an intramolecular reaction once the P2 primer is hybridized and extended by a DNA polymerase. An experiment described herein, and the results shown in FIGS. 13B-C(curves and data labeled by black dots) indicate that this ‘P2-P3 coupled-primers method provides for an appreciable acceleration of PCR (amplification power of 2.5), similar to that observed in the case of the uncoupled-primers method (see FIGS. 5, 6, and 8, curves and data labeled by black dots). Likewise, application of step 4 during PCR cycles further accelerates amplification (amplification power of 5.4, FIGS. 13B and 13C). Use of a ‘conventional’ PCR profile that does not incorporate incubation of the reaction mixture at a ‘sufficient’ temperature of 76° C. (curves and data labeled by empty diamond symbols 0) showed a very modest level of acceleration that is likely caused by relatively slow heat-ramping of the instrument through the sufficient temperature range. The same phenomenon was also observed in the P2-P3-overlapping primers' experiments (see FIGS. 10A-B and 11A-B).

### Exemplary Four-Primer Accelerated PCR Embodiment

The three-primer method embodiments of the invention may be further extended by addition of a fourth primer P4. Such a four-primer embodiment (in this instance, non-overlapping) is illustrated in FIGS. 2A and 2B, wherein the P3 and P4 primers hybridize to the respective target strands at positions 5′ upstream from the hybridized P2 and P1 primers, respectively.

FIG. 2A illustrates, according to particular exemplary aspects of the present invention, amplifying, in a first PCR cycle, the target nucleic acid sequence of FIG. 1 using four oligonucleotide primers P1, P2, P3, and P4. Similar to FIG. 1A, FIG. 2A shows the amplification scheme for two complementary strands of the target nucleic acid sequence (solid lines) with adjacent contiguous (indefinite ends) (dashed lines). In stage A, primers P1 and P2 hybridize to complimentary primer binding sites on the first and the second target strand sequence, respectively, and are extended by the DNA polymerase resulting in initial double-stranded products having 3′-sequences of indefinite length. In stage B, oligonucleotide primers P3 and P4 hybridize to complementary primer binding sites on the second and first target sequence strands, respectively, at positions located 5′ upstream from the hybridized P2 and P1 primer extension products, respectively. DNA polymerase extends the hybridized primers P3 and P4 toward (e.g., up to) the 5′-ends of the P2 and P1 primer extension products (produced during stage A), which in this instance may be long and highly thermostable due to their indefinite 3′-ends. In analogy with the scheme of FIG. 1A, initiation of melting of these particular initial P2 and P1 extension products does not occur at the “sufficient temperature”, and thus the initial thermostable P2 and P1 extension products preclude the P3 and P4 primer extension products from extending beyond the 5′-end of the P2 and P1 primer extension products, respectively. Subsequent denaturation of all primer extension products by incubating the reaction mixture at the denaturation temperature in stage C recovers the single stranded first and second target strands with indefinite ends and produces P1 and P2 primer extension products having indefinite ends. According to particular aspects of the present invention, formation of these two single-stranded products during denaturation after the first cycle of PCR provides for accelerated PCR in the next, successive cycle of the PCR, as illustrated in FIG. 2B. The blocked initial P3 and P4 primer extension products produced in this first PCR cycle do not incorporate P1 and P2 binding sites, respectively, and thus are effectively wasted because they cannot participate in further amplification during subsequent PCR cycles. These ‘incomplete’ P3 and P4 extension products are shown in FIG. 2A with a “X” at their 3′-ends.

FIG. 2B shows a next, successive cycle of the PCR. The P2 primer extension product with the indefinite 3′-sequence incorporates P1 and P4 primer binding sites (reaction scheme shown on the left of FIG. 2B), whereas the P1 primer extension product with its indefinite 3′-sequence incorporates P2 and P3 binding sites (reaction scheme shown on the right of FIG. 2B). As illustrated in FIG. 2B for convenience, the PCR cycle is presented as being divided into two parallel processes, which in analogy to the PCR cycle of FIG. 1B, proceed through steps 1, 2, 3A, and 3B. Unlike the situation in stage B of FIG. 2A, the P1 and P2 primer extension products in the PCR cycle of FIG. 2B are not long and indefinite, and rather have definite, relatively short lengths, incorporating P2 and P1 binding sites at their 3′-ends, respectively. According to particular aspects of the present invention, incubation of the reaction mixture at the sufficient temperature (lower than the denaturation temperature) initiates thermal melting of these P1 and P2 primer extension products, and where the hybridized P4 and P3 primer extension products have thermal stabilities sufficient to provide for their further extension at the sufficient temperature to produce double-stranded, further extended P4 and P3 primer extension products incorporating P2 and P1 primer binding sites at their 3′ ends (step 3B). This is accompanied by provision of the P1 and P2 primer extension products in single stranded form at the end of steps 3B. According to further aspects of the present invention, the P1 primer extension product hybridized to the P2 primer extension product produced in the first PCR cycle has nearly the same thermal stability as the P2 primer extension product hybridized to the P1 primer extension product produced at step 1 and is therefore also melted at the ‘sufficient’ temperature. In particular exemplary aspects of the present invention the PCR cycle and amplification can be terminated at the completion of step 3B by increasing the reaction temperature to the denaturation temperature, where all hybridized primer extension products are denatured-effectively bypassing step 4. In preferred exemplary aspects and similar to the methods of FIGS. 1A-1C, however, in order to further increase the PCR amplification power, the reaction temperature is lowered in step 4 of this next successive cycle of the PCR, to a temperature below the sufficient temperature to enable hybridization of additional primers P1 and P2 to the single-stranded extension product of these primers produced in step 3. Subsequent extension of these complexes by DNA polymerase in step 4 results in double stranded amplification products incorporating P1 and P2 primer binding sites at their 3′ ends. According to yet further aspects of the present invention, when step 4 is included, the P2 and P1 primer extension products are amplified twice (once prior to incubating the reaction mixture at the sufficient temperature, and once thereafter) during this next, successive cycle of the PCR. Upon completion of step 4, the reaction temperature is raised to the denaturation temperature to denature all double stranded amplification products rendering them single stranded for a further successive cycle of the PCR. As in the three-primer embodiment of FIGS. 1A-1C, the number of the short amplicons framed by the P1 and P2 primer sequences grows at a rate much faster than doubling in each cycle. Amplification of these short products strongly accelerates PCR because many (or most, or substantially all) of them are replicated twice in each consecutive PCR cycle because of the use of the sufficient temperature, lower than the denaturation temperature, to initiate melting of the P2 primer extension products (and of the P1 primer extension products having P2 primer binding sites at their 3′-ends).

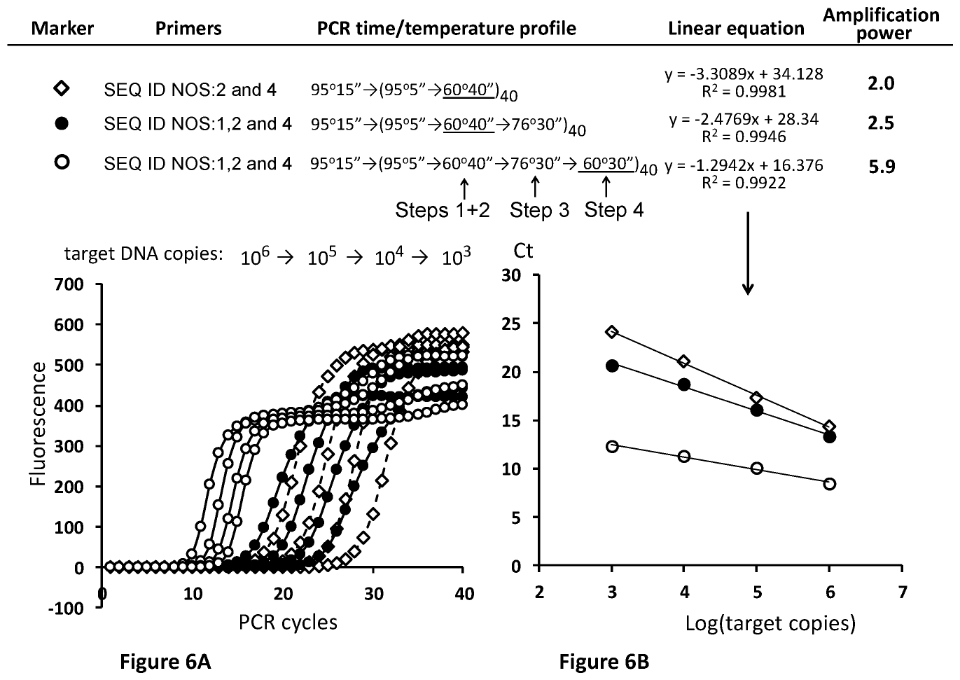

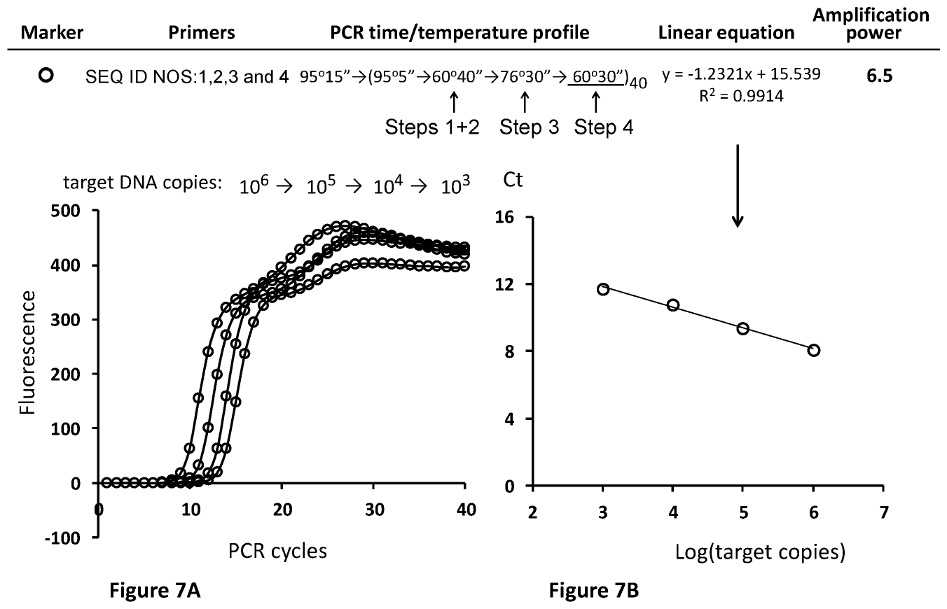

According to additional aspects of the present invention, therefore, this four-primer accelerated PCR embodiment further increases the amplification power compared to the three-primer accelerated PCR embodiment of FIGS. 1A-1C (e.g., compare results of FIGS. 5 and 6 (three primers, with those of FIG. 7 (four primers)). The four-primer method provides for amplification of two long primer extension products framed by the sequences of P1, P3, and P2, P4 primers, respectively. In subsequent cycles, both of these long amplicons continuously supply short amplicons into the PCR reaction, thus further accelerating PCR in contrast to the three-primer method of FIGS. 1A-1C, wherein only one long amplification product incorporating P1 and P3 binding sites at its 3′-ends serves as the reaction accelerator. According to yet further aspects of the present invention, while a double-stranded amplification product having a P3 and a P4 binding site at its 3′-ends is disfavored by the reaction scheme of FIGS. 2A and 2B and does not appear in a detectable concentration at the beginning of PCR, such a product may begin to accumulate during late cycles.

In the methods, the four-primer accelerated PCR embodiments may utilize all the variants of three-primer design illustrated in FIGS. 1A-C, 9 and 12. For example, all four primers can be designed/selected as shown in each of these figures. Moreover, the three-primer methods are inter-compatible. For example, the P2 and P3 primers may be covalently coupled as illustrated in FIG. 12, and/or the the P1 and P4 primers may be uncoupled and designed according to methods of FIGS. 1A-C or, alternatively, FIG. 9.

As will be appreciated from the above description, therefore, use of a fourth P4 primer in the methods can further accelerate PCR. Experimental results from a working Example using the above-described four-primer embodiment are shown in FIG. 7, wherein a substantial, and unprecedented amplification power of 6.5 has been achieved.

### Kits for Accelerated PCR

As discussed above, yet further embodiments of the invention provide accelerated PCR kits, comprising three or four oligonucleotide primers having a system design, in terms of sequence, hybridization/thermal stability properties, and spatial positioning with respect to strands of a target sequence, to provide for primer extension products consistent with performing the accelerated PCR methods of the invention, as discussed and illustrated in exemplary FIGS. 1A-1C, 2A-2B, 9 and 12.

### Discussion of Particular Factors and Conditions for Accelerated PCR Methods and Kits

Relative thermal stability of primers. According to particular aspects, an important factor for methods and kits of the invention is the difference in hybridization properties (thermal stability; Tm) between the P2 primer extension product incorporating a P1 primer binding site at its 3′-end, and the P3 primer and/or its extension product, particularly the intermediate P3 primer extension product blocked at the 5′-end of the downstream hybridized P2 primer extension product prior to incubation of the reaction mixture at the “sufficient temperature” (the temperature that is sufficient to initiate thermal melting of the hybridized second primer extension product).

In the methods (and kits), therefore, an important condition for achieving accelerated PCR amplification is the thermal stability of the P3 primer and/or the intermediate third primer extension product at the “sufficient temperature”. The better hybridization property (thermal stability; Tm) of the P3 primer and/or of its intermediate extension product at the sufficient temperature, the greater the achievable amount of PCR acceleration. Stabilization of the hybridized P3 primer and/or of its intermediate extension product relative to that of the second primer extension product may be accomplished in a number of ways. First, for example, if possible, the primer binding sites and/or target sequence may be selected such that the P2 primer extension product is relatively A,T-rich and/or the third primer or its intermediate extension product is relatively G,C-rich, particularly within its 3′-sequence adjacent the hybridized and extended second primer. Unfortunately, however, this is commonly not an option and to nonetheless illustrate the broad applicability of the methods the target sequence chosen for the exemplary working Examples provided herein was actually selected to be relatively G,C-rich with respect to the P1 and P2 primer extension products (although the 6407-mer long M13mp18 sequence provided ample opportunity for better target sequence selection in this regard).

A second strategy is appropriate spatial positioning (appropriate distancing) of the third primer binding site from that of the second primer on the second target strand. In general, the longer the intermediate third primer product, the more thermostable it can be. The distancing approach, although effective for increasing thermal stability, can result in increased overall PCR time. In the methods, when the P2 and P3 primers hybridize to the target strand close or next (immediately adjacent) to each other, or when their binding sites overlap, P3 primer hybridization properties (thermal stability) can be elevated by use of appropriately long sequences as exemplified by the experiments of FIGS. 10A-B and 11A-B. An alternative and/or complementary approach is to elevate the P3 primer hybridization properties (thermal stability; Tm) by incorporating polymerase-compatible duplex-stabilizing modifications into its structure, which also stabilizes the intermediate P3 primer extension product. For example, the P3 primer may carry 5′-conjugated intercalators (e.g., Lokhov, S. G., et al. (1992) or minor groove binding moieties (e.g., Hedgpeth, J., et al. (2010) U.S. Pat. No. 7,794,945), or comprise 5-methyl cytosine (5-MeC) and 2,6-diamino-purine (2-amA) nucleotide analogs in place of cytosine and adenine, respectively (e.g., Lebedev, Y., et al., 1996).

A further alternative and/or complementary approach to modify the relative hybridization dynamics (e.g., the relative thermal stability; Tm) of the P3 primer extension product is to destabilize the P2 primer extension product. As mentioned above, wherever possible, primer binding sites and/or the target sequence may be selected to produce a relatively A,T-rich P2 primer extension product incorporating a P1 primer binding site at it 3′-end.

A yet further approach is to design/select appropriate spatial positioning of the P2 and P1 primers, i.e., the length of the amplicon bracketed by the P2 and P1 primers. While methods of the invention generally place no limits on the length of the P2 primer extension product, reduction of the nucleotide sequence length of this amplification product is perhaps the most effective way to control its thermal stability (Tm). The nucleotide distance between the P2 primer sequence and the P1 binding site located at the 3′-end of the P2 primer extension product can be sufficiently long (e.g., 20 nucleotides, or perhaps even greater), for example, to incorporate a binding site for a FRET-labeled oligonucleotide probe such as “Molecular Beacons” (Lizardi, P. M., et al., 1992), “Eclipse Probes” (Afonina, I. A., et al., 2002), or a probe segment of Scorpion primers (e.g., Whitcombe, D., et al., 1999). However, this distance may be shorter than 20 nucleotides, 15 nucleotides or shorter, preferably 10 nucleotides or shorter, and even more preferably 5 nucleotides or shorter, or 3 nucleotides or shorter. In the working examples provided herein (the primers and results using them are shown in FIGS. 3, 5-8, 10, 11 and 13), the nucleotide distance between the P2 primer sequence and the P1 binding site is zero. In particular, this exemplary primer design of P1, P2, and P3 primers renders the P2 primer extension product incorporating a P1 primer binding site at its 3′-end relatively unstable to facilitate initiation of its melting at the “sufficient temperature” (75-81.5° C. in these examples), whereas the P3 primer extension product has an adequate (to provide for further extension of the P3 primer extension product) thermal stability at that ‘sufficient temperature’, wherein these factors result in an elevated amplification power of PCR as illustrated (FIGS. 3, 5-8, 10, 11 and 13).

In yet further design aspects, destabilization (reduced thermal stability) of the hybridized P2 primer extension product (e.g., amplicon bracketed by the P2 and P1 primers) can be provided by use of polymerase-compatible duplex-destabilizing modifications in the design of P1 and P2 primers. For example, 7-deaza purine nucleotide analogs (Seela, et al., 1992), deoxyinosine, or deoxyuridine nucleotides (Kawase, Y., et al., 1986, Martin, F. H., et al., 1985) may be used for this purpose. According to additional aspects, this does not exclude the use of FRET effects for multiplex detection of more than one target sequence in a sample, since FRET-labelling can be transferred to the primer design using art-recognized technologies, e.g., Nazarenko, I. A., et al., U.S. Pat. No. 5,866,336; Rabbani, E., et al., U.S. Pat. No. 9,353,405. A preferred strategy in the design of FRET-labeled PCR primers was applied in the working Example 4 provided herein, the primers and results of which are illustrated in FIGS. 3 and 8.

Relative primer extension timing dynamics. An additional P2 and P3 primer design factor may be considered to attain maximal amplification power in the accelerated PCR methods of the invention; namely relative primer extension timing dynamics (e.g., timing of primer extension events). Ideally, to avoid interference of P2 primer extension by upstream P3 primer extension events, the P2 and P3 primers are selected/designed such that the P2 primer hybridizes to its binding sites and then extends (or sufficiently extends) before the P3 primer extension product reaches and extends over the P2 primer binding site. Such timing dominance in hybridization and extension of the P2 primer over that of the P3 primer during the accelerated PCR methods may be achieved, for example, by applying one of or a combination of ‘distancing,’ ‘thermodynamic,’ and ‘kinetic’ factor approaches.

For example, in a thermodynamic approach, the P2 primer can be designed to have better hybridization properties (e.g., greater thermal stability) than the P3 primer, to provide a thermodynamic-based timing advantage. In this case, after full denaturation to initiate a PCR cycle, the reaction temperature may be reduced to a level at which both P2 and P3 primers can hybridize, but where the P2 primer would hybridize first and get extended before the P3 primer extension product reaches and extends over the P2 primer binding site. Alternatively, the reaction mixture may be first incubated at a temperature at which the P2 primer hybridizes and becomes extended, and then at a lower temperature to provide for hybridization of the P3 primer. While such thermal stability advantage approaches can provide for timing/temporal dominance of P2 hybridization and primer extension, it may not always be practical in accelerated PCR system design, where the melting temperature of the hybridized P2 primer extension product incorporating P1 binding site at its 3′-end (e.g., the P2/P1 amplicon), must be low enough so that melting is initiated at the designed/selected “sufficient temperature.”

The P1 primer, in theory, may be designed/selected in length and nucleotide composition to hybridize and become extended at any stage of the accelerated PCR cycle. The hybridization properties of both the P1 and P2 primers, however, determine the thermal stability of the hybridized P2 extension product (the P2/P1 amplicon), and therefore the hybridization properties of the P1 primer are preferably designed to perform at the lowest temperature of the accelerated PCR cycle, or at least to be consistent with) those of the P2 primer in the overall system design. For example, in the working Examples provided herein (the results of which are shown in FIGS. 3 and 5-8) all three primers (P1, P2, and P3) have nearly the same hybridization properties or melting temperatures (FIG. 3). In this system design, therefore, the timing dominance of the P2 primer hybridization and extension over that of the P3 primer is controlled by using a ‘distancing’ effect approach (relative spatial positioning within the target sequence), wherein increased distancing of the P3 primer binding site from the P2 binding site (i) helps to stabilize the P3 primer extension product as discussed above, and (ii) provides the P2 primer with a timing/temporal advantage for hybridization and extension before the P3 primer extension product reaches and extends over the P2 primer binding site on the second target strand. Use of appropriate distancing between the P3 and P2 primer binding sites, therefore, provides a timing/temporal/kinetic benefit, and further provides for design flexibility in terms of thermal stability of the P3 primer. In methods of the invention, for example, given a P2 primer timing dynamic and/or kinetic advantage provided by distancing, a P3 primer can be designed/selected to have a greater thermal stability in forming a complementary duplex with the target sequence relative to that of the complementary complex of a P2 primer with the target sequence.

Alternatively, and according to further aspects of the invention, the temporal dominance of the P2 primer hybridization and extension over that of the P3 primer can be modulated by a ‘kinetic’ factor, wherein the P2 primer is present in the PCR reaction mixture at a concentration that is higher than that of the P3 primer. For purposes of system design, the magnitude of the difference in concentration between the P2 and P3 primers depends on the magnitude of the difference in their respective hybridization properties with the target sequence. Kinetic approaches can be combined with distancing and/or with thermodynamic approaches for fine tuning the reaction dynamics. For example, distancing may be zero (e.g. FIGS. 9-11) where the kinetic and/or thermal stability advantages suffice to satisfy the overall system design/performance, or distancing may be designed/selected to incrementally complement kinetic and/or thermal stability factors. In system designs wherein ‘kinetic’ factors are primarily in control of the temporal dominance of primer hybridization and extension, the P2 primer concentration in the reaction mixture is preferably twice that of the P3 primer, but may be adjusted to fine-tune the overall reaction dynamics. As illustrated in the working Examples (the results of which are shown in FIGS. 5-8), however, appropriate distancing between P2 and P3 primers can be a sufficiently effective factor on its own for establishing temporal dominance, without use of the ‘thermodynamic’ and/or ‘kinetic’ factors, and can provide for very substantial amplification powers.

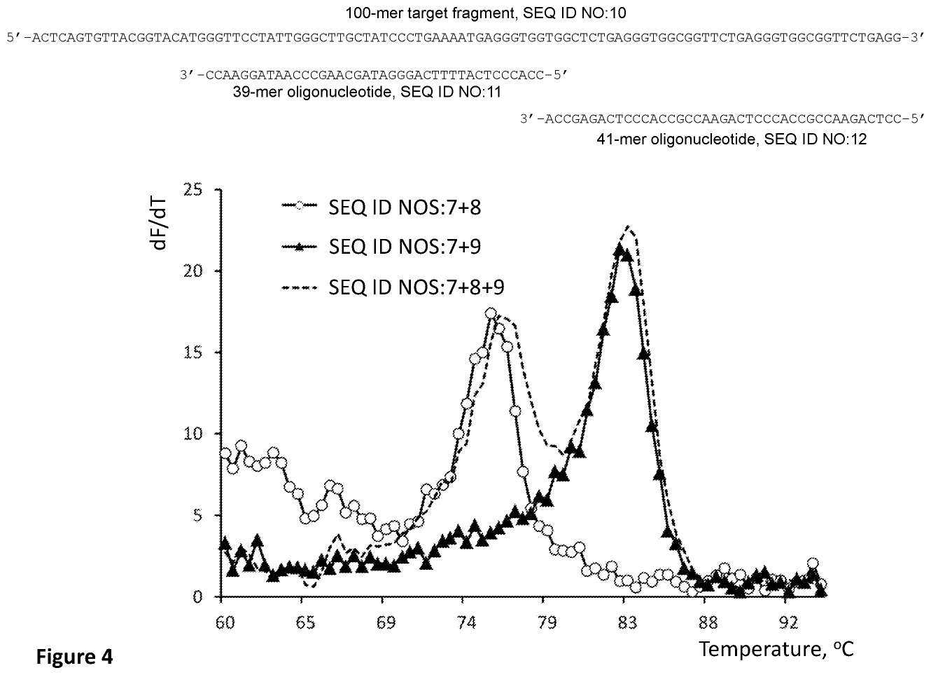

According to yet further aspects of the invention, however, the distancing factor approach can be ignored. For example, the experiments of the working Examples (the results of which are shown in FIGS. 5-8) was performed using a 41-mer oligonucleotide SEQ ID NO:12 shown in FIG. 4 as an alternative P3 primer. This long alternative P3 primer hybridizes to the target strand immediately adjacent the P2 primer (SEQ ID NO:2, FIG. 3) with no distancing, yet it is sufficiently stable (without any elongation) at the “sufficient temperature” (see FIG. 4). Although the P2 primer (SEQ ID NO:2) has a substantially lower hybridization property (thermal stability; Tm) than that of the 41-mer primer SEQ ID:12 (Tm's of 64° C. vs 83° C., respectively), the 100-fold excess of the P2 primer over the P3 primer (2,000 nM vs. 20 nM) provided the temporal dominance (‘kinetic’ factor) of P2 primer hybridization and extension, over that of the P3 primer, resulting in substantial acceleration of PCR as illustrated by the experiments of FIGS. 10A-B. The same ‘kinetic’ factor was successfully applied in the experiment of FIGS. 11A-B, wherein the P3 primer binding site completely overlaps the P2 primer binding site. The ‘kinetic’ factor can be effectively applied in the method embodiments of FIGS. 1, 2 and 9, but not in the method embodiment of FIG. 12, due to the covalent coupling of P2 and P3 primers wherein they present in the reaction mixture at equal concentration. However, by appropriately spacing their respective binding sites, these primers can be balanced in their hybridization properties as discussed above, resulting in substantial acceleration of PCR (e.g., see FIGS. 13A-C). Regarding the ‘kinetic’ factor, it was found that use of P1 or P2, or preferably both of these primers at elevated concentrations (e.g. greater than 200 nM, preferably greater than 500 nM, or even more preferably greater than 800 nM) can be beneficial for acceleration of PCR in all methods, including those method embodiments of FIGS. 1,2, 9 and 12. According to particular aspects of the invention, while the working examples (e.g., FIGS. 5-8, 10, 11 and 13) show that the accelerated PCR methods can provide vastly improved amplification power (e.g., up to 6.5) relative to that achieved in the prior art, appropriate system design using the approaches, factors and parameters disclosed herein can provide even greater amplification powers, and the present working Examples do no place a limit on the maximum amplification power that can be achieved using the accelerated PCR methods of the invention. According to further aspects, in certain instances it may nonetheless be desirable to limit the amplification power to an accelerated level/value less than the maximum achievable level to maximize accuracy for determining the absolute amount of a target nucleic acid initially present in a sample. Accordingly, in the accelerated PCR methods of the invention, the amplification power may be controlled, (e.g., raised or lowered, by using variations of numerous assay parameters (e.g., absolute and/or relative concentration of oligonucleotide primers, hybridization properties of the primers, distancing, time and/or temperature at particular steps of PCR cycle (e.g., manipulation in time/temperature profile), use of one or more particular DNA polymerase(s), dNTP concentrations, salt (and particularly magnesium ion concentration(s)), pH of the solution, etc.

Moreover, as described above, in particular aspects of the accelerated PCR methods using three primers (FIG. 1B), the amplification products can be denatured after completion of step 3A and 3B, effectively bypassing step 4. As illustrated in FIGS. 5 and 6, while this truncated aspect (step 4 bypass) substantially accelerates PCR, reducing the number of cycles to detect the same amount of target nucleic acid relative to conventional PCR, incorporation of step 4 into the time/temperature profile vastly accelerates PCR, although accompanied by an attendant increase in overall time of the assay. In contrast to art-recognized, classical strand-displacement techniques (see, e.g. U.S. Pat. No. 5,270,184 to Walker G. T. et al; U.S. Pat. No. 6,214,587 to Dattagupta N. et al; U.S. Pat. No. 5,854,033 to Lizardi P.), the present inventive methods are based on, e.g., melting of P2 and/or P1 primer extension product incorporating P1 and P2 binding site at their 3′-ends, respectively, at the “sufficient temperature” during amplification resulting in accelerated PCR. However, to the extent that a classical strand-displacement mechanism might yet occur to some low degree (e.g., in the reaction schemes of FIGS. 1 and 2), it would only benefit the methods of the invention.

In further aspects, as described herein, further amplification power can be achieved by inclusion of a fourth P4 primer (see reaction scheme of FIGS. 2A and 2B, and the results of FIG. 7), although with some degree of attendant increased complexity and primer costs.

The disclosed accelerated PCR methods and kits have broad target applicability in view of the ample system design approaches and options as discussed in detail herein.

### Working Examples

The following working Examples are provided and disclosed for illustrative purposes to demonstrate exemplary embodiments of the accelerated PCR methods of the invention for amplification and detection of target nucleic acids, and are not intended to limit the scope of the inventive methods, kits and applications.

### Example 1

### Materials and Methods

Synthesis of Oligonucleotide Components. Structures and sequences of an exemplary M13mp18 target sequence SEQ ID NO:6 was detected using various PCR primers (SEQ ID NOS:1-4) and a FRET primer (SEQ ID NO:5) as shown in FIG. 3.

FIGS. 3, 4 and 13A show, according to particular exemplary aspects of the present invention, fragments of M13mp18 vector sequence (target DNA sequence, SEQ ID NOS:6, 10 and 14) and nine 2′-deoxyribo oligonucleotide primers (SEQ ID NOS:1-4, 7-9, 12 and 13) used in the working Examples provided herein. Oligonucleotide SEQ ID NO:5 is a FRET-modified primer that is an analog of primer SEQ ID NO:2 incorporating a 5′-conjugated 6-fluorescein dye (FAM) and Black Hole Quencher dye (BHQ1) that was internally linked to the 5-base position of deoxyribo uridine nucleoside (shown as ‘U’ bolded). The oligonucleotide sequences are aligned with the target DNA in the orientation (5′-to-3′) as indicated. The melting temperatures (Tm's) were calculated for a corresponding full complement duplex (200 nM) in 50 mM NaCl, 3 mM MgCl₂, 1.2 mM dNTPs using OligoAnalyzer™ program provided on internet by Integrated DNA Technology (see the IDT website).

For the FRET-labeled primer, a 6-fluorescein reporting dye was incorporated onto the 5′-end, and a BHQ1 “dark” quencher was introduced to the middle of the primer (SEQ ID NO:5) using respective phosphoramidites from Glen Research (Cat. NO:10-1963-xx and 10-5941-xx). Standard phosphoramidites, ‘reversed’ phosphoramidites and Spacer C18 for synthesis of P2-P3-coupled primers (SEQ ID NOS:13 and 14, FIG. 13A), solid supports and reagents to perform the solid support oligonucleotide synthesis were also purchased from Glen Research. 0.25 M 5-ethylthio-1H-tetrazile solution was used as a coupling agent. Oligonucleotides were synthesized either on AB1394 DNA synthesizer (Applied Biosystems) or MerMaid 6™ DNA synthesizer (BioAutomation Corporation) using protocols recommended by the manufacturers for 0.2 micromole synthesis scales. After the automated synthesis, oligonucleotides were deprotected in aqueous 30% ammonia solution by incubation for 12 hours at 55.degree. C. or 2 hours at 70° C.

Tri-ON oligonucleotides were purified by HPLC on a reverse phase C18 column (LUNA 5 μm, 100 A, 250×4.6 mm, Phenomenex Inc.) using a gradient of acetonitrile in 0.1 M triethyl ammonium acetate (pH 8.0) or carbonate (pH 8.5) buffer with flow rate of 1 ml/min. A gradient profile including washing stage 0→14% (10 sec), 14→45% (23 min), 45→90% (10 min), 90→90% (5 min), 90→0% (30 sec), 0→0% (7 min) was applied for purification of all Tri-ON oligonucleotides. The product-containing fractions were dried down in vacuum (SPD 1010 SpeedVac™, TermoSavant) and trityl groups were removed by treatment in 80% aqueous acetic acid at room temperature for 40-60 min. After addition to the detritylation reaction (100 μl) of 20 μl sodium acetate (3 M), the oligonucleotide components were precipitated in alcohol (1.5 ml), centrifuged, washed with alcohol and dried down. Concentration of the oligonucleotide components was determined based on the optical density at 260 nm and the extinction coefficients calculated for individual oligonucleotides using on-line OligoAnalyzer™ 3.0 software provided by Integrated DNA Technologies. Based on the measurements, convenient stock solutions in water were prepared and stored at −20° C. for further use. The purity of all prepared oligonucleotide components was confirmed by analytical 8-20% PAAG electrophoresis, reverse phase HPLC and by spectroscopy on Cary 4000 UV-VIS spectrophotometer equipped with Cary WinUV software, Bio Package 3.0 (Varian, Inc.). Oligodeoxyribonucleotides SEQ ID NOS: 9-12 (shown in FIGS. 3 and 4) were purchased from Integrated DNA Technologies and dissolved in water amounts recommended by the manufacturer for each oligonucleotide to provide stock solutions of 100 micro molar concentration.

### SEQUENCE LISTING

(primer)
SEQ ID NO: 1
5′-GGTTCCTATTGGGCTTGCT-3′
(primer)
SEQ ID NO: 2
5′-CCACCCTCATTTTCAGGGAT-5′
(primer)
SEQ ID NO: 3
5′-CCTCAGAACCGCCA-5′
(primer)
SEQ ID NO: 4
5′-GTTGTCTGTGGAATGCTACA-3′
(FRET labeled primer)
SEQ ID NO: 5
5′-(FAM)CCACCCTCATT(BHQ1-U)TCAGGGAT-3′
(fragment of target sequence M13mp18)
SEQ ID NO: 6
5′-GAGGGTTGTCTGTGGAATGCTACAGGCGTTGTAGTTTGTACTGGTGAC
GAAACTCAGTGTTACGGTACATGGGTTCCTATTGGGCTTGCTATCCCTGAA
AATGAGGGTGGTG GCTCTGAGGGTGGCGGTTCTGAGGGTGGCGGTTCTGA
GGG-3′
(primer)
SEQ ID NO: 7
5′-ACTCAGTGTTACGGTACATGGGTTCCTATTGGGCT TGCT-3′
(primer)
SEQ ID NO: 8
5′-ACCCTCAGAGCCACCACCCTCATTTTCAGGGAT-3′
(primer)
SEQ ID NO: 9
5′-CTCAGAACCGCCACCCTCAGAACCGCCACCCTCAGAGCCACCACCCTC
ATTTTCAGGGAT-3′
(fragment of target sequence M13mp18 used in
melting experiments)
SEQ ID NO: 10
5′-ACTCAGTGTTACGGTACATGGGTTCCTATTGGGCTTGCTATCCCTGAA
AATGAGGGTGGTGGCTCTGAGGGTGGCGGTTCTGAGGGTGGCGGTTCTGAG
G-3′
(fragment of target sequence M13mp18 used in
melting experiments)
SEQ ID NO: 11
5′-CCACCCTCATTTTCAGGGATAGCAAGCCCAATAGGAACC-3′
(fragment of target sequence M13mp18 used in
melting experiments)
SEQ ID NO: 12
5′-CCTCAGAACCGCCACCCTCAGAACCGCCACCCTCAGAGCCA-3′
(first primer portion of composite oligonucleotide
comprising two primers coupled through their 5′-
ends with linker
—O—PO(OH)—[O—(CH₂CH₂O)₆—PO(OH)]₃—O—)
SEQ ID NO: 13
5′-CCACCCTCATTTTCAGGGAT-3′
(second primer portion of composite oligonucleotide
comprising two primers coupled through their 5′-
ends with linker
—O—PO(OH)—[O—(CH₂CH₂O)₆—PO(OH)]₃—O—)
SEQ ID NO: 14
5′-ATCACCGTACTCA-3′
(fragment of target sequence M13mp18)
SEQ ID NO: 15
5′-TGGGTTCCTATTGGGCTTGCTATCCCTGAAAATGAGGGTGGTGGCTCT
GAGGGTGGCGGTTCTGAGGGTGGCGGTTCTGAGGGTGGCGGTACTAAACCT
CCTGAGTACGGTGATACA-3′

### Example 2

### Primer Melting Temperatures were Determined

This example describes nucleic acid melting experiments to measure the thermal stability of exemplary oligonucleotides hybridized to an M13mp18 target sequence to simulate the hybridization properties of a P2 primer extension product incorporating a P1 primer binding site at its 3′-end, and a P3 primer extension product extended up to the 5′-end of P2 primer extension product according to the reaction scheme shown in FIG. 1A, 1B, 2A, and 2B.

FIG. 4 shows the sequences of the oligonucleotides (SEQ ID NOS:10-12) melted in presence of EvaGreen™ fluorescent dye and the results of these melting experiments (melting graph). Reaction mixtures of 25 μl total volume were prepared to incorporate the following components, amounts and concentrations: 100-mer oligonucleotide (SEQ ID NO:10)—220 nM; 39-mer and 41-mer oligonucleotides (SEQ ID NOS:11,12)—200 nM when present; 0.2 units of EvaGreen™ dye (Biotium, Cat. NO:31000); Bovine Serum Albumin (New England BioLabs Inc., Cat. NO:B9000S)—100 ng; dNTPs (Roche Custombiotech, Cat. NO:04920171103)—300 nM each in 50 mM KCl, 3 mM MgCl₂, 20 mM Tris-HCl (pH 8.0). The reaction mixtures were dispensed into wells of the plastic PCR plate (96-Well Non-Skirted PCR Plates, Multimax, Cat. NO: T-3069-1), sealed using 8× Strip Optical Caps (Agilent Technologies, Cat. NO:401425), placed into the heating block of Stratagene Mx3005P instrument for real time PCR (Agilent Technologies), heated to 95° C. (5 seconds), cooled down and incubated at 60° C. for 4 minutes, and then slowly heated to 95° C. using the instrumental software which constantly recorded the fluorescence of the reaction wells in EvaGreen channel. The data were transferred into Microsoft Excel and are shown in FIG. 4 in a form of first derivative of the corresponding melting curves (dF/dT) versus the reaction temperature.

Each melting curve shown in FIG. 4 is an average of 5 parallel reactions of the same composition. The results show that the product of extension (SEQ ID NO:11) of the primer P2 (SEQ ID NO:2) incorporating the primer P1 (SEQ ID NO:1) binding site at its 3′-end that is less thermostable (Tm=76° C.) than the product of extension (SEQ ID NO:12) of primer P3 (SEQ ID NO:3) up to the 5′-end of the extension product of P2 (Tm=83° C.). The data indicates that the melting peak of the P3 primer extension product is abnormally asymmetric and this oligonucleotide (SEQ ID NO:12) initiates melting nearly at the same temperatures as the oligonucleotide SEQ ID NO:11, but at a slower rate.

Specifically, FIG. 4 shows, according to particular exemplary aspects of the present invention, sequences of oligonucleotides used in melting experiments described in detail herein in Example 2. The graph in FIG. 4 shows the results of these melting experiments provided in the form of a first derivative of the corresponding fluorescence melting curves (Y-axis) plotted against the reaction temperature (dF/dT) on the X-axis. Oligonucleotide SEQ ID NO:11 represents a P2 primer extension product (see SEQ ID NO:2, FIG. 3) that incorporates a P1 primer (SEQ ID NO:1) binding site at its 3′-end. Oligonucleotide SEQ ID NO:12 represents a P3 primer (SEQ ID NO:3, FIG. 3) extension product of extension of P3 primer (SEQ ID NO:3, FIG. 3) up to the 5′-end of the P2 primer extension product. Both of these primer extension products are produced in the exemplary PCR experiments provided herein (and are shown FIGS. 1 and 2) and the melting curves of FIG. 4 allow comparison of their relative thermal stabilities with a 100-mer target sequence (SEQ ID NO:10; a portion of SEQ ID NO:6)). As it evident from the melting results, the complementary complex of SEQ ID NO:11+SEQ ID NO:10 is less stable (maximum at approx.76° C.) than the complementary complex of SEQ ID NO:12+SEQ ID NO:10 (maximum at approx. 83° C.), such that the P3 primer extension product (SEQ ID NO:12) hybridized to the complementary target strand has a thermal stability at the ‘sufficient’ temperature (e.g., at 76° C. at which melting of the P2 primer extension product (SEQ ID NO:11) is initiated. When both primer extension products (SEQ ID NOS:11 and 12) are hybridized to complementary sequence SEQ ID NO:10 and subsequently melted (dashed line), a slight stabilization of the P2 primer extension product was observed. This is due to an art-recognized effect (e.g., Pyshnyi D. V., et al., 2003, J. Biomol. Struct. Dyn., V. 21, No:3, 459-467) wherein a more stable duplex stabilizes a less stable duplex if these duplexes are in a coaxial stacking (i.e., two oligonucleotides hybridized to a single-stranded polynucleotide next to each other, without a nucleotide gap between them). In the working Examples and results thereof as shown in FIGS. 5-8 and 10, this stabilization effect was ignored due to its relatively minor contribution/scale. According to additional aspects, however, in method embodiments with a less thermally stable P2 primer extension product than that shown in FIG. 4, the coaxial stacking stabilization may be considered and included to the system design.

### Example 3

### A Circular Double-Stranded Cloning Vector M13mp18 was Linearized

The target nucleic acid used in the exemplary PCR experiments provided herein was selected from the sequence cloning vector M13mp18, which in its double-stranded form is a covalently closed, circular 7249-base pair DNA. Circular DNAs are very resistant to denaturation unless they linearized, e.g. by restriction nucleases. A reaction mixture of 50 μl of volume was prepared to contain 1 μg of M13mp18 RF I DNA (New England BioLabs, Cat. NO: N4018S), 20 U of EcoR1 endonuclease (New England BioLabs, Cat. NO: R0101S), 1×NEBuffer (supplied with the enzyme). After 1-hour incubation at 37° C., the linearized vector was diluted in 20 mM Tris-HCl (pH8) buffer to prepare appropriate stock solutions with DNA concentrations variable in orders of magnitude scale.

### Example 4

### Exemplary Accelerated PCR Methods were Performed

The PCR reactions provided herein were prepared on ice by mixing the reagent stock solutions. Unless otherwise indicated, all reaction mixtures incorporated 50 mM KCl, 3 mM Mg(SO₄)₂, 20 mM Tris-HCl (pH8), 300 μM each of four 2′-deoxyribonucleoside 5′-triphosphates (dNTPs: dATP, dTTP, dCTP and dGTP), 0.1 mg/ml Bovine Serum Albumin (New England BioLabs, Cat. NO:B9000S), 0.2 U/μl (FIGS. 5-7) or 1 U/μl (FIGS. 10, 11 and 13) of EvaGreen™ fluorescent dye, when present (Biotium, Cat. NO:31000) and 0.08 U/μl (FIGS. 5-8) or 0.2 U/μl (FIGS. 10 and 11) of Top DNA polymerase (Bioneer, Cat. NO:E-3101-2). Vent(exo-) DNA polymerase (New England BioLabs, Cat. NO:M0257S) was used in the experiments of FIGS. 13B-C at a reaction concentration of 0.05 U/μl. Sequences and structures of oligonucleotide primers used in real time PCR experiments are shown in FIGS. 3, 4 and 13A, PCR time/temperature cycle profile are indicated in the corresponding figure descriptions elsewhere herein, describing every particular experiment (FIGS. 5-8, 10, 11 and 13). All primers were used at a concentration of 200 nM in the experiments of FIGS. 5-8 and 13 whereas, in the experiments of FIGS. 10 and 11, P1 and P2 primer concentrations (SEQ ID NOS:1, 2, 7 and 8) was increased to 2000 nM while P3 primer concentration (SEQ ID NOS:9 and 12) was reduced to 20 nM. Circular double-stranded cloning vector M13mp18, linearized as described in Example 3, was used as a sequence of interest (target sequence). Fluorescence monitoring during PCR was conducted using either a SmartCycler™ (Cepheid Corporation, FIGS. 5-8) or Magnetic Induction Cycler™ (Bio Molecular Systems, FIGS. 10, 11 and 13) real time instruments. The fluorescence curves shown in FIGS. 5-8, 10, 11 and 13 are an average of at least four parallel reactions. The data (plotted as fluorescence vs. PCR cycle) were transferred into an Excel™ format (Microsoft Corporation). The fluorescent curve threshold value (Ct) was determined as the cycle number at which the semi-log of fluorescence of the curve reached value of 1 (SmartCycler™) or 0.03 (Magnetic Induction Cycler™).

The M13mp18-derived target sequence selected for the exemplary experiments was intentionally selected to be a relatively ‘difficult’ sequence to amplify and detect due to an elevated ˜50-60% G/C content. Nonetheless, a significantly enhanced amplification power of 2.5 was reached in the working Examples employing three primers (SEQ ID NOS:1, 2, and 4 (P1, P2, and P4, respectively) and a PCR profile excluding step 4 (black dots in FIGS. 5 and 6). A slightly lower amplification power of 2.4 was seen in the case of using primer P3 SEQ ID NO:3 (in place of P4), and can be explained by a marginal thermal stability at the sufficient temperature (76° C.) of the product of extension of this primer up to the 5′-end of primer P2 SEQ ID NO:2 hybridized to the target. As evident from the melting results, the product of extension of primer P3 SEQ ID NO:3 (oligonucleotide SEQ ID NO:12, FIG. 4) also initiates melting at the sufficient temperature along with the extension product of primer SEQ ID NO:2 (oligonucleotide SEQ ID NO:11, FIG. 4), but at a much lower rate. This result indicates that further thermal stabilization of the P3 primer extension product will be needed by, e.g., further distancing of the P3 and P2 primer binding sites on the target strand.

Inclusion of step 4 (see, e.g., step 4 as shown in FIGS. 1B and 2B) into the PCR time/temperature profile vastly accelerates the amplification, resulting in an amplification power of 5.1 and 5.9 (FIGS. 5 and 6, respectively). Note, however, that parameters of this step may be varied, and the duration of this step 4 may be reduced or, alternatively, the temperature can be elevated without noticeable reduction in the system amplification power (not shown).

FIGS. 5A and 5B show, according to particular exemplary aspects of the present invention, results of EvaGreen™ fluorescence monitoring during conventional PCR in comparison with accelerated PCR using a three-primer embodiment of the invention (SEQ ID NO:1 as P1 primer, SEQ ID NO:2 as P2 primer and SEQ ID NO:3 as P3 primer, FIG. 3). Three groups of the curves are shown. The fluorescence curves labeled by empty diamond symbols (0) correspond to a conventional PCR format using only two primers (SEQ ID NOS:1 and 3), whereas the curves labeled by circle symbols correspond to the accelerated PCR method of the invention using three primers (SEQ ID NOS:1, 2, and 3) with step 4 applied (open circles; o) or without step 4 applied (filled circles; •) in the PCR profile. The four curves within each group differ only by the target reaction load (initial amount of target DNA) that was varied in order of magnitude increments from left to right as indicated. A fluorescence threshold for each curve of FIG. 5A was determined and plotted (using the same marker symbols) versus logarithm of the target loads in FIG. 5B. The data points of FIG. 5B display good linear trend with an R² value >0.99. The slopes of the linear equations were used to calculate an amplification power coefficient for each case. These coefficients (typically rounded to tenths), the linear equations obtained from FIG. 5B, primer numbers and abbreviated PCR time/temperature profiles used in the reactions are listed at the top of FIGS. 5A and 5B and identified by corresponding marker symbols. For example, the time/temperature profile abbreviation 95° 15″→(95° 5″→60° 40″→76° 30″→60° 30″)₄₀ means the reaction mixture was incubated at 95° C. for 15 seconds followed by 40 cycles comprising incubation at 95° C. for 5 seconds, then at 60° C. for 40 seconds (step 1+2), followed by incubation for 30 seconds at 76° C. (the “sufficient temperature” in this example) (step 3), and then 30 seconds at 60° C. (step 4). The fluorescence detection stage is underlined. All figures relating to real time PCR experiments (FIGS. 5-8, 10-11 and 13B-C) follow the same pattern of data presentation. Any significant differences or deviations are discussed below or outline in the attendant text.

FIGS. 6A and 6B show, according to particular exemplary aspects of the present invention, results of EvaGreen™ fluorescence monitoring during conventional PCR, in comparison with accelerated PCR using a three-primer method embodiment of the invention. The experiments of FIGS. 6A and 6B are identical in all aspects, including the reaction composition, PCR setup and data presentation to those shown in FIGS. 5A and 5B, but primer SEQ ID NO:3 used in FIGS. 5A and 5B as the third P3 primer was replaced by primer SEQ ID NO:4 (see FIG. 3) as indicated.

FIGS. 7A and 7B show, according to particular exemplary aspects of the present invention, results of EvaGreen™ fluorescence monitoring during accelerated PCR using a four-primers' method embodiment of the invention (SEQ ID NOS:1-4, see FIG. 3) and a PCR time/temperature profile incorporating step 4 as indicated. The reaction composition and PCR setup as well as the data presentation are otherwise identical to those shown in FIGS. 5 and 6. Fluorescence threshold for each curve of FIG. 7A was determined and plotted versus logarithm of the target loads in FIG. 7B. The slope coefficients of the linear equations were used to calculate the PCR amplification power in this particular case. An amplification power of 6.5 was observed in the experiment employing four primers with step 4 incorporated into the PCR profile (FIG. 7). Analysis of the linear equation in this case in comparison with that for the conventional PCR format (FIG. 5, empty diamonds) quantify the considerable reduction, relative to conventional PCR, in the number of PCR cycles (by ˜18-20 cycles) required for detecting the same amount of the target sequence in a sample.

FIGS. 8A and 8B show, according to particular exemplary aspects of the present invention, detection of accelerated PCR amplified material in real time using a FRET-labelled P2 primer. The reaction mixtures in FIG. 8A had the same composition and time/temperature profile as those marked by filled circles (•) in FIG. 5A, but P2 primer SEQ ID NO:2 was replaced by its FRET-labelled analog SEQ ID NO:5 (see structures in FIG. 3) and EvaGreen™ fluorescent dye was omitted. For the exemplary experiments, in order to reduce thermal stability of the hybridized P2 primer extension product incorporating the binding site of P1 primer at it 3′-end, these primers were selected such that their binding sites on corresponding strands of the target sequence are positioned next to each other without overlap. Although this primer design provides the shortest possible, and therefore unstable duplex, it leaves no space for integration of a FRET-probe. The results of FIG. 8, however, illustrate an effective option for FRET-detection of the amplified material for such ‘extreme’ design cases. Primer P2 was labeled with two FRET dyes (FIG. 3, SEQ ID NO:5) and used in place of unlabeled primer of the same sequence (SEQ ID NO: 2) in the shown experiment employing three primers (P1, P2(FRET), and P3) with no step 4 involved. PCR acceleration was observed in this case, although the amplification power of 2.3 was slightly lower than 2.4 calculated for the case using an unlabeled primer SEQ ID NO:2 (filled circles, FIG. 5). The most likely reason is the incorporation of the BHQ1 quenching dye near the middle of the primer oligonucleotide chain, representing a structural modification that may be moderately polymerase-compatible. However, the experiments nonetheless support applicability of methods for multiplex detection of numerous target nucleic acids in the same reaction mixture.

FIGS. 10A and 10B show, according to particular exemplary aspects of the present invention, results of EvaGreen™ fluorescence monitoring during PCR using a three-primer method embodiment. In contrast to the experimental data shown in FIGS. 5-8 for embodiments wherein the P3 primer hybridizes to the target strand at a position 5′ upstream from the hybridized second primer extension product, the results of FIGS. 10A and 10B illustrate another embodiment wherein the P3 primer hybridizes to the target strand immediately adjacent the 5′-end of the hybridized P2 primer, without a nucleotide gap. A 41-mer oligonucleotide (SEQ ID NO:12, FIG. 4) was used as the P3 primer, whereas the P1 and P2 primers (SEQ ID NOS:1 and 2) were the same as in FIGS. 5-7. This 41-mer P3 primer represents a product of extension of a 14-mer primer (SEQ ID NO:3) up to the 5′-end of the P2 primer (SEQ ID NO:2) hybridized to the same target strand, as would be synthesized during accelerated PCR of FIG. 5. As discussed in Example 2 above, this 41-mer oligonucleotide forms a very stable duplex with target strand (Tm=83° C., FIG. 4), whereas the estimated melting temperature of the P2 primer (SEQ ID NO:2) was much lower (61° C.; FIG. 3). In order to compensate for this thermodynamic disadvantage of the P2 primer and promote its dominance in hybridization kinetics over the P3 primer, a ‘kinetic’ factor was introduced in the experiments of FIG. 10. Reaction concentrations of P2 and P1 were increased in these experiments to 2000 nM, whereas the concentration of the P3 primer was reduced to 20 nM. Experimental parameters such as the reaction composition and PCR setup as well as the data presentation are otherwise identical to those shown in FIGS. 5-8. A target-dilution experiment representing a ‘conventional’ PCR was performed (fluorescence curves labeled by empty diamond symbols (0), FIG. 10A), wherein the reaction mixture incorporated all three P1, P2 and P3 primers, but the ‘conventional’ PCR profile did not incorporate incubation at a ‘sufficient’ temperature of 75° C. However, because the reaction mixture is exposed for a short time period to a sufficient temperature range during the instrumental heat-ramping, a very minor but still detectable PCR acceleration was observed (amplification power of 2.03). The experiments representing the ‘conventional’ PCR were performed the same way in FIGS. 11 and 13, and the same phenomenon was observed in both cases. Incorporation of an incubation at a ‘sufficient’ temperature in the reaction time/temperature profile (curves labeled by filled circles; •), and addition of step 4 (open circles; ◯) led to strong PCR acceleration (amplification power of 2.8 and 6.1, respectively).

FIGS. 11A and 11B show, according to particular exemplary aspects of the present invention, results of EvaGreen™ fluorescence monitoring during PCR using a three-primer method embodiment of the invention. This experiment in many aspects is similar to that of FIG. 10 in which the P3 primer (SEQ ID NO:9) binding site incorporates the binding site sequence of P2 primer (SEQ ID NO:8, FIG. 3) as an extreme example of P2/P3 primer design. In the embodiment of FIGS. 11A and 11B, the P2 and P3 primers may overlap partially, e.g. one or more nucleotides. As in the case of the example of FIG. 10, the thermal stability of a very long, 60-mer P3 primer (Tm=81° C.) surpasses that of its counterpart P2 primer (Tm=74.5° C., FIG. 3). Likewise, to compensate the thermodynamic disadvantage of the P2 primer and promote its dominance in hybridization kinetics over the P3 primer during the instrumental cool-ramping, the reaction concentrations of P2 and P1 were increased to 2000 nM, whereas the concentration of the P3 primer was reduced to nM. Moreover, the reaction performed using the ‘conventional’ PCR profile (curves labeled by empty diamond symbols (0), FIGS. 11A-B) displayed a very weak PCR acceleration for the same reasons as discussed above. Incorporation of incubation at a ‘sufficient’ temperature in the reaction time/temperature profile (curves labeled by filled circles; •) and addition of step 4 (open circles; ∘) led to strong PCR acceleration (amplification power of 3.0 and 5.7, respectively) comparable with the PCR efficiency observed in the experiments of FIG. 10, as well as in FIGS. 5-8.

FIGS. 13B and 13C show results of EvaGreen™ fluorescence monitoring during accelerated PCR and illustrate yet additional exemplary aspects of the present invention based on a three-primer method. In contrast to the approach of FIGS. 1A-C, the P2 and P3 primers used in the methods of FIG. 12, are covalently coupled to each other. The relative concentrations of P2 and P3 primers cannot, therefore, be manipulated to provide a ‘kinetic’ factor; that is, they are always equal because of the covalent coupling. Other remaining factors, such as ‘thermodynamics’ and ‘primer-distancing’ effect, can nonetheless be used to control the acceleration power of PCR in this method embodiment. The primer-distancing requirements are similar to those in the methods of FIGS. 1A-C, and the product of extension of the P3 primer up to the 5′-end of P2 primer hybridized to the same target strand may yet have an adequate hybridization property (Tm), primarily provided by its length, to remain bound at the ‘sufficient’ temperature of the accelerated PCR. Covalent coupling of P2 and P3 primers may insure the dominance in hybridization kinetics of P2 primer to the target strand. An example of such a primer design is shown in FIG. 13A (SEQ ID NO:13). In this case, the P3 primer is coupled to the P2 primer through a long non-hybridizing linker, has very weak hybridization properties (Tm=46.4° C.) compared to the P2 primer (Tm=61.4° C.), yet still performs well at the annealing temperature of 62° C. (see FIGS. 13B-C) because of intramolecular reaction kinetics. Once P2 primer hybridizes to the target strand and is extended by DNA polymerase, then hybridization and extension of the P3 primer to the 5′-upstream target site becomes effective as an intramolecular reaction, unambiguously defining the order of P2 and P3 primers' performance in PCR. FIGS. 13B and 13C show results of EvaGreen™ fluorescence monitoring during accelerated PCR using a P1 primer (SEQ ID NO:1) and a P2/P3-covalently coupled (via a linker) hybrid primer (SEQ ID NOS:13 and 14, FIG. 13A). The real-time PCR experiments, bypassing step 4 (curves labeled by filled circles; •) and incorporating step 4 (open circles; ∘), showed the level of reaction acceleration (amplification power of 2.5 and 5.4, respectively), which is consistent with the level of acceleration reported here for the other exemplary method embodiments (see FIGS. 5-8, 10 and 11).

### REFERENCES CITED, AND INCORPORATED HEREIN BY REFERENCE THERETO FOR THEIR RESPECTIVE RELEVANT TEACHINGS

Afonina, I. A., et al. (2002), BioTechniques, V. 32, 940-949.
Ausubel, F. M., et al., eds. (1993), Current Protocols in Molecular Biology, Vol. 1, Chapter 2, Section I, John Wiley & Sons, New York.
Beaucage, S. L., and Caruthers, M. H. (1981), Tetrahedron Lett. V. 22, 1859-1862.
Boom, W. R., Henriette, M. A., Kievits, T., Lens, P. F. (1993) U.S. Pat. No. 5,234,809.
Breslauer, K. J., et al (1986) Proc. Natl. Acad. Sci. USA, V. 83, 3746-3750.
Caruthers, M. H., Matteucci, M. D. (1984) U.S. Pat. No. 4,458,066.
Dattagupta N., Stull P. D., Spingola M., and Kacian D. L. (2001) U.S. Pat. No. 6,214,587.
Didenko, V. V. (2001) BioTechniques, V. 31, 1106-1121.
Duck, P., Bender R., Crosby W., Robertson J. G. (1989) U.S. Pat. No. 4,876,187.
Eckstein, F., ed., (1991), Oligonucleotides and Analogs: A Practical Approach. Oxford University Press, New York.
Eftink, M. R. (1991), Fluorescence quenching: theory and applications. In Lakowicz, J. R. (ed.), Topics in Fluorescence Spectroscopy. Plenum Press, New York, V. 2: 53-126.
Forster, T. (1965), Delocalized excitation and excitation transfer. In Sinanoglu, O. (ed.), Modern Quantum Chemistry, Istanbul Lectures, part Ill. Academic Press, New York, 93-137.
Gait, M. J., ed., (1984), Oligonucleotide Synthesis: A Practical Approach, IRL Practical Approach Series, IRL Press, Oxford.
Ge, W., et al. (2012), US Patent Application Publication 2012/0058481.
Harris, C. L., et al. (2013), Biotechniques, V. 54, No. 2, 93-97.
Heller, M. J., and Morrison, L. E. (1985) Chemiluminescent and fluorescent probes for DNA hybridization. In Kingsbury, D. T., and Falkow, S. (eds.), Rapid Detection and Identification of Infectious Agents. Academic Press, New York, 245-256.
Higuchi, R. (2018) U.S. Pat. Appl., Pub. No: US2018/0066304 A1.
Ignatov, K. B., et al. (2014) Biotechniques, V. 57, No. 2, 81-87.
Kawase, Y., et al. (1986) Nucleic Acids Res., V. 14, 7727-7736.
Kornberg, A., and Baker, T. (1992) DNA Replication, Second Edition, W. H. Freeman and Company, New York.
Hedgpeth, J., Afonina, I. A., Kutyavin, I. V., Lukhtanov, E. A., Belousov, E. S., Meyer, Jr., R. B. (2010) U.S. Pat. No. 7,794,945.
Kutyavin, I. V., Milesi, D., Hoekstra, M. F. (2007) U.S. Pat. No. 7,252,940.
Kutyavin, I. V. (2007a) PCT patent application, WO/2007/127999.
Kutyavin, I. V. (2007b) PCT patent application, WO/2007/127992.
Lebedev, Y., et al. (1996) Genet. Anal., V. 13, 15-21.
Lehninger, A. L. (1975) Biochemistry, 2nd edition. New York, Worth Publishers, Inc.
Livak, K. J., Flood, S. J. A., Marmaro, J. and Mullah, K. B. (1998) U.S. Pat. No. 5,723,591.
Lizardi P. (1998) U.S. Pat. No. 5,854,033.
Lizardi, P. M., et al. (1992) U.S. Pat. No. 5,118,801.
Lokhov, S. G., et al. (1992) Bioconjugate Chem., V. 3, No. 5, 414-419.
Mackay, I. M., et a0.1 (2002) Nucleic Acids Res., V. 30, 1292-1305.
Mackay, J., and Landt, O. (2007) Methods Mol. Biol., V. 353, 237-262.
Martin, F. H., et al. (1985) Nucleic Acids Res., V. 13, 8927-8938.
McPherson, M. J., et al., eds (1991) PCR: A Practical Approach. IRL Press, Oxford.
McPherson, M. J., et al., eds (1995) PCR2: A Practical Approach. IRL Press, Oxford.
Miller, S. A., Dykes, D. D., Polesky, H. F. (1988) Nucleic Acids Res., V. 16, 1215.
Mullis, K. B. (1987) U.S. Pat. No. 4,683,202.
Mullis, K. B., et al. (1987) U.S. Pat. No. 4,683,195.
Nazarenko, I. A., et al. (1999) U.S. Pat. No. 5,866,336.
Pyshnyi, D. V., et al., 2003, J. Biomol. Struct. Dyn., V. 21, No:3, 459-467.
Rabbani, E., et al. (2016) U.S. Pat. No. 9,353,405.
Robelek, R., Niu, L., Schmid, E. L., Knoll, W. (2004) Anal. Chem., V. 76, 6160-6165.
Sambrook, J., Fritsch, E. F., and Maniatis, T. (1989) Molecular Cloning: A Laboratory Manual, 2nd Edition. Cold Spring Harbor Lab. Cold Spring Harbor, N.Y.
SantaLucia, J., Jr. (1998) Proc. Natl. Acad. Sci. USA, V. 95, 1460-1465.
Seela, et al., (1992) Nucleic Acids Research, vol. 20, 55-61.
Walker G. T., Little M. C., and Nadeau J. G. (1993) U.S. Pat. No. 5,270,184.
Walsh, P. S., Metzger, D. A., and Higuchi, R. (1991) Biotechniques, V. 10, 506-513.
Whitcombe, D., et al. (1999) Nature Biotech., 17:804-807.

1. A method for accelerated polymerase chain reaction (PCR) amplification, comprising performing PCR in a suitable reaction mixture containing DNA polymerase, a nucleic acid target sequence and at least three oligonucleotide primers each complementary to a respective primer binding site of the target sequence and each present in excess relative to the target sequence, wherein at least one cycle of the PCR includes
producing a first primer (P1) extension product hybridized to a first strand of the target sequence and having a binding site for a second oligonucleotide primer (P2) and for a third oligonucleotide primer (P3);
producing a P2 extension product hybridized to a second strand of the target sequence and having a P1 primer binding site at its 3′ end; and
incubating the reaction mixture at a temperature sufficient to initiate thermal melting of the hybridized P2 extension product, and producing, at the sufficient temperature, a full-length P3 primer extension product hybridized to the second strand of the target sequence and having a sequence complementary to the P2 binding site, and having a P1 binding site at its 3′ end, wherein the P2 primer extension product is not hybridized to the second strand of the target sequence and is accessible to be primed by another P1 primer, and wherein the sufficient temperature is less than a denaturation temperature used to initiate a next, successive cycle of the PCR.

2. The method of claim 1, wherein the at least one cycle of the PCR comprises:
hybridizing the P1 primer to the first strand of the target sequence and extending the hybridized P1 primer to provide the P1 primer extension product hybridized to the first strand of the target sequence and having the binding site for the P2 primer and for the P3 primer;
hybridizing the P2 primer to the second, complementary strand of the target sequence and extending the hybridized P2 primer to provide the P2 primer extension product hybridized to the second strand of the target sequence and having the P1 primer binding site at its 3′ end;
hybridizing the P3 primer to the second strand of the target sequence at a position 5′ upstream from the hybridized P2 primer extension product, and extending the hybridized P3 primer toward the 5′-end of the hybridized P2 primer extension product to provide a partial P3 primer extension product hybridized to the second strand of the target sequence and lacking the P1 primer binding site at its 3′ end; and
incubating the reaction mixture at the temperature sufficient to initiate thermal melting of the hybridized P2 primer extension product, wherein the hybridized partial P3 primer extension product has a thermal stability sufficient to provide for its further extension at the sufficient temperature, and further extending the hybridized partial P3 primer extension product to provide the full-length P3 primer extension product hybridized to the second strand of the target sequence.

3. The method of claim 1, wherein the at least one cycle of the PCR comprises:
hybridizing the P1 primer to the first strand of the target sequence and extending the hybridized P1 primer to provide the P1 primer extension product hybridized to the first strand of the target sequence and having the binding site for the P2 primer and for the P3 primer;
hybridizing the P2 primer to the second, complementary strand of the target sequence and extending the hybridized P2 primer to provide the P2 primer extension product hybridized to the second strand of the target sequence and having the P1 primer binding site at its 3′ end;
incubating the reaction mixture at the temperature sufficient to initiate thermal melting of the hybridized P2 primer extension product; and
hybridizing the P3 oligonucleotide primer to the second strand of the target sequence at a position immediately adjacent the 3′-end of the P2 primer binding site, or at a position overlapping the P2 primer binding site, and extending, at the sufficient temperature, the hybridized P3 primer to provide the full-length P3 primer extension product hybridized to the second strand of the target sequence.

4. The method of any one of claims 1-3, further comprising, in the at least one cycle of the PCR, hybridizing another P1 primer to the P2 primer extension product not hybridized to the second strand of the target sequence, and extending the hybridized other P1 primer to provide a P1/P2 double-stranded extension product having P1 and P2 primer binding sites at its 3′ ends.

5. The method of claim 4, further comprising, after forming the P1/P2 double-stranded extension product, incubating the reaction mixture at a denaturation temperature greater than the sufficient temperature to denature all hybridized primer extension products including the P1 primer extension product having the P2 and the P3 primer binding sites, the full-length P3 primer extension product, and the P1/P2 double-stranded extension product.

6. The method of claim 5, further comprising in a next, successive cycle of the PCR:
hybridizing additional P1 and P2 primers to respective primer binding sites of the denatured primer extension products from the preceding cycle, including to the respective primer binding sites of the denatured strands of the P1 primer extension product, of the full-length P3 primer extension product, and of the P1/P2 double-stranded extension product;
extending the hybridized additional P1 and P2 primers to provide additional hybridized P1 and P2 primer extension products, including an additional P2 primer extension product hybridized to the P1 primer extension product, an additional P1 primer extension product hybridized to the P3 primer extension product, and additional P1/P2 double-stranded extension products having additional P1 and P2 primer binding sites at their 3′ ends;
incubating the reaction mixture at the sufficient temperature to initiate thermal melting of the additional hybridized P2 primer extension products, including of the additional P2 primer extension product hybridized to the P1 primer extension product, and of the additional P1/P2 double-stranded extension products;
hybridizing yet additional P1 and P2 primers to respective primer binding sites of the thermally-melted additional P2 primer extension products, including to respective primer binding sites of the thermally melted strands of the additional P2 primer extension product hybridized to the P1 primer extension product, and of the additional P1/P2 double-stranded extension products; and
extending the hybridized yet additional P1 and P2 primers to provide yet additional P1/P2 double-stranded extension products having yet additional P1 and P2 primer binding sites at their 3′ ends, wherein the P1/P2 double-stranded extension product produced in the preceding at least one cycle of the PCR is amplified twice in this successive cycle of the PCR, once prior to incubating the reaction mixture at the sufficient temperature, and once thereafter.

7. The method of claim 6, wherein at least one of the yet additional P1/P2 double-stranded extension products is derived from the P1 primer extension product of the preceding at least one cycle of the PCR.

8. The method of claim 6 or 7, comprising hybridizing additional P3 primers to respective primer binding sites of the denatured primer extension products from the preceding at least one PCR cycle that have P3 primer binding sites, and extending, at the sufficient temperature, the hybridized additional P3 primer extension products to produce additional full-length P3 primer extension products.

9. The method of claim 8, further comprising, incubating the reaction mixture at the denaturation temperature to denature all hybridized primer extension products.

10. The method of claim 9, further comprising, in a further successive cycle of the PCR, twice amplifying at least one, more than one, or substantially all of the yet additional P1/P2 double-stranded extension products.

11. The method of any one of claims 4-10, wherein, in the at least one cycle of the PCR, the hybridizing another P1 primer to the P2 primer extension product not hybridized to the second strand of the target sequence is performed at a lower reaction temperature than the sufficient temperature.

12. The method of claim 2, wherein, in the at least one cycle of the PCR, hybridizing the P3 primer to the second strand of the target sequence is performed at an identical, different, or lower reaction temperature than a temperature used for hybridizing the P2 primer to the second strand of the target sequence.

13. The method of any one of claims 1-12, wherein, in the at least one cycle of the PCR, the hybridized P3 primer, or the hybridized partial P3 primer extension product, has a greater thermal stability than that of the hybridized P2 primer extension product having a P1 primer binding site at its 3′ end.

14. The method of any one of claims 1-13, wherein upon completion of the PCR, the number of P2 primer extension products is greater than that of the P3 primer extension products, at least in part because the P2 primer extension products are amplified twice in one or in each of a plurality of cycles of the PCR.

15. The method of claim 1-14, wherein upon completion of the PCR, the ratio of the number of P2 primer extension products to that of the full-length P3 primer extension products is determined, at least in part, by at least one of: the distance between the second and third primer binding sites on the second strand of the target sequence; the relative concentrations of the second and third primers; or by the relative thermal stability of the complementary duplexes of the second and the third primers with their respective binding sites.

16. The method of any one of claims 1-15, wherein the concentration of the P2 primer is greater than that of the P3 primer.

17. The method of claim 2, wherein the thermal stability of the complementary duplex of the P2 primer with its binding site is greater than that of the complementary duplex of the P3 primer with its binding site.

18. The method of claim 2, further comprising a fourth oligonucleotide primer (P4) complementary to a respective primer binding site of the target sequence and present in excess relative to the target sequence, wherein the at least one cycle of the PCR includes hybridizing the P4 primer to the first strand of the target sequence at a position 5′ upstream from the hybridized P1 primer extension product, and extending the hybridized P4 primer toward the 5′-end of the hybridized P1 primer extension product to provide a partial P4 primer extension product hybridized to the first strand of the target sequence and lacking a P2 primer binding site at its 3′ end, wherein the sufficient temperature is sufficient to initiate thermal melting of the hybridized P1 primer extension product, and wherein the hybridized P4 primer extension product has a thermal stability sufficient to provide for its further extension at the sufficient temperature; and
further extending the hybridized P4 primer extension product to produce a full-length P4 primer extension product hybridized to the first strand of the target sequence and having a P2 primer binding site, and wherein the P1 primer extension product is not hybridized to the first strand of the target sequence, and is accessible to priming by another P2 oligonucleotide primer.

19. The method of any one of claims 1-18, wherein the distance, in nucleotides, between the 5′ end of P1 primer binding site on the first strand and the 5′ end of the P2 primer binding site on the second strand is less than 20, less than 15, less than 10, less than 5, less than 4, less than 3, less than 2, 1, or 0, or is a value in the range of 0 to 20, or in any subrange thereof.

20. The method of claim 19, wherein the distance is 0 to 3 nucleotides.

21. The method of any one of claims 1-20, wherein an amplification power of at least 2.2 is provided.

22. The method of claim 21 wherein an amplification power of at least 2.5 is provided.

23. The method of any one of claims 1-22, wherein the P1 primer, the P2 primer, or both incorporate at least one polymerase-compatible duplex-destabilizing modification.

24. The method of any one of claims 1-23, wherein the P3 primer incorporates at least one polymerase-compatible duplex-stabilizing modification.

25. The method of claim 18, wherein the P1 primer, the P2 primer, or both incorporate at least one polymerase-compatible duplex-destabilizing modification, and wherein the P3 primer, the P4 primer, or both incorporate at least one polymerase-compatible duplex-stabilizing modification.

26. The method of any one of claims 1-25, wherein the amplification products are detected.

27. The method of claim 26, wherein the amplification and detection reactions are performed simultaneously, in real time.

28. The method of claim 27, further comprising determining the amount of the target nucleic acid in or from a sample.

29. The method of claim 28, wherein the reaction mixture further comprises a detectable label.

30. The method of claim 29, wherein the detectable label comprises a fluorescent label.

31. The method of claim 30, wherein the reaction mixture comprises an oligonucleotide probe labeled with two dyes that are in FRET interaction, and wherein duplex formation of the probe with products of extension of the P1 or the P2 primers disrupts FRET resulting in a detectable signal.

32. The method of claim 30, wherein at least one of the P1 and the P2 primers is labeled with two dyes that are in a FRET interaction, and wherein hybridization and extension of the at least one labeled primer during PCR disrupts the FRET interaction resulting in a detectable signal.

33. The method of any one of claims 1, 2, 4-32, wherein the P2 and the P3 primers are covalently coupled to each other.

34. The method of claim 33, wherein the P2 and the P3 primers are covalently coupled at their 5′-ends.

35. The method of claim 34, wherein the P2 and the P3 primers are coupled through a linker.

36. The method of claim 35, wherein the linker comprises a oligoethylene glycol moiety.

37. A PCR kit, comprising at least three oligonucleotide primers each complementary to a respective primer binding site of a target sequence, wherein a first oligonucleotide primer (P1) is complementary to a P1 primer binding site on a first strand of the target sequence, wherein the second oligonucleotide primer (P2) is complementary to a P2 primer binding site on a second, complementary strand of the target sequence to define a P1/P2 amplicon sequence of the target sequence, wherein the third oligonucleotide primer (P3) is complementary to a P3 primer binding site on the second strand of the target sequence, and wherein, relative to the target sequence, the sequences and relative positions of the P2 and third P3 binding sites on the second strand of the target sequence are configured such that thermal stability of a P3 primer, or of a P3 primer extension product extending to the 3′-end of the second primer binding site is greater than that of a P2 primer extension product having a P1 primer binding site at its 3′-end.

38. The PCR kit of claim 37, wherein the P3 primer binding site on the second strand of the target sequence is at a position 3′ downstream from the P2 primer binding site.

39. The PCR kit of claim 38, wherein the P2 and the P3 primers are covalently coupled to each other.

40. The PCR kit of claim 39, wherein the P2 and the P3 primers are covalently coupled at their 5′-ends.

41. The PCR kit of claim 40, wherein the P2 and the P3 primers are coupled through a linker.

42. The PCR kit of claim 41, wherein the linker comprises a oligoethylene glycol moiety.

43. The PCR kit of any one of claims 37-42, wherein the distance, in nucleotides, between the 5′ end of P1 primer binding site on the first strand and the 5′ end of the P2 primer binding site on the second strand is less than 20, less than 15, less than 10, less than 5, less than 4, less than 3, less than 2, 1, or 0, or is a value in the range of 0 to 20, or in any subrange thereof.

44. The PCR kit of claim 43, wherein the distance is 0 to 3 nucleotides.

45. A PCR kit, comprising at least three oligonucleotide primers each complementary to a respective primer binding site of a target sequence, wherein a first oligonucleotide primer (P1) is complementary to a P1 primer binding site on a first strand of the target sequence, wherein a second oligonucleotide primer (P2) is complementary to a P2 primer binding site on a second, complementary strand of the target sequence to define an P1/P2 amplicon sequence of the target sequence, wherein a third oligonucleotide primer (P3) is complementary to a P3 primer binding site on the second strand of the target sequence, and wherein, relative to the target sequence, the distance, in nucleotides, between the 5′ end of P1 primer binding site on the first strand and the 5′ end of the P2 primer binding site on the second strand is less than 20, less than 15, less than 10, less than 5, less than 4, less than 3, less than 2, 1, or 0, or is a value in the range of 0 to 20, or in any subrange thereof.

46. The PCR kit of claim 45, wherein the distance is 0 to 3 nucleotides.

47. The PCR kit of claim 45 or 46, wherein the P3 primer binding site on the second strand of the target sequence is at a position 3′ downstream from the P2 primer binding site.

48. The PCR kit of claim 47, wherein the P2 and the P3 primers are covalently coupled to each other.

49. The PCR kit of claim 48, wherein the P2 and the P3 primers are covalently coupled at their 5′-ends.

50. The PCR kit of claim 49, wherein the P2 and the P3 primers are coupled through a linker.

51. The PCR kit of claim 50, wherein the linker comprises a oligoethylene glycol moiety.

52. The PCR kit of any one of claims 45-51, wherein, relative to the target sequence, the sequences and relative positions of the P2 and the P3 primer binding sites on the second strand of the target sequence are such that thermal stability of a P3 primer, or of a P3 primer extension product extending to the 3′-end of the P2 primer binding site is greater than that of a P2 primer extension product having a P1 primer binding site at its 3′-end.


SEQUENCE LISTING (15 sequences)

SEQ ID NO: 1 - DNA, 19 bp, Artificial Sequence
  primer 1
ggttcctattgggcttgct

SEQ ID NO: 2 - DNA, 20 bp, Artificial Sequence
  primer 2
ccaccctcattttcagggat

SEQ ID NO: 3 - DNA, 14 bp, Artificial Sequence
  primer 3
cctcagaaccgcca

SEQ ID NO: 4 - DNA, 20 bp, Artificial Sequence
  primer 4
gttgtctgtggaatgctaca

SEQ ID NO: 5 - DNA, 20 bp, Artificial Sequence
  FRET primer
  misc_structure (12)..(12): n = BHQ1-U
  misc_feature (12)..(12): n is a, c, g, or t
ccaccctcattntcagggat

SEQ ID NO: 6 - DNA, 152 bp, Artificial Sequence
  M13MP18 sequence portion
gagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgaggg

SEQ ID NO: 7 - DNA, 39 bp, Artificial Sequence
  primer 7
actcagtgttacggtacatgggttcctattgggcttgct

SEQ ID NO: 8 - DNA, 33 bp, Artificial Sequence
  primer 8
accctcagagccaccaccctcattttcagggat

SEQ ID NO: 9 - DNA, 60 bp, Artificial Sequence
  primer 9
ctcagaaccgccaccctcagaaccgccaccctcagagccaccaccctcattttcagggat

SEQ ID NO: 10 - DNA, 100 bp, Artificial Sequence
  fragment of target sequence M13mp18 used in melting experiments
actcagtgttacggtacatgggttcctattgggcttgctatccctgaaaatgagggtggtggctctgagggtggcggttctgagggtggcggttctgagg

SEQ ID NO: 11 - DNA, 39 bp, Artificial Sequence
  fragment of target sequence M13mp18 used in melting experiments-2
ccaccctcattttcagggatagcaagcccaataggaacc

SEQ ID NO: 12 - DNA, 41 bp, Artificial Sequence
  fragment of target sequence M13mp18 used in melting experiments-3
cctcagaaccgccaccctcagaaccgccaccctcagagcca

SEQ ID NO: 13 - DNA, 20 bp, Artificial Sequence
  first primer portion of composite oligonucleotide representing two primers coupled through their 5′-ends
ccaccctcattttcagggat

SEQ ID NO: 14 - DNA, 13 bp, Artificial Sequence
  second primer portion of composite oligonucleotide representing two primers coupled through their 5′-ends
atcaccgtactca

SEQ ID NO: 15 - DNA, 117 bp, Artificial Sequence
  fragment of target sequence M13mp18
tgggttcctattgggcttgctatccctgaaaatgagggtggtggctctgagggtggcggttctgagggtggcggttctgagggtggcggtactaaacctcctgagtacggtgataca